(19) United States
(12) Patent Application Publication
(10) Pub. No.: US 2010/0093609 A1
(43) Pub. Date: Apr. 15, 2010

(54) PRODRUGS OF TRICIRIBINE AND TRICIRIBINE PHOSPHATE

(76) Inventors: John Hilfinger, Ann Arbor, MI (US); Wei Shen, Dearborn, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/593,709

(22) PCT Filed: Mar. 31, 2008
(86) PCT No.: PCT/US08/58862

§ 371 Date: Sep. 29, 2009

Related U.S. Application Data
(60) Provisional application No. 60/908,824, filed on Mar. 29, 2007.

(51) Int. Cl. A61K 38/14; C07H 19/04; C07K 9/00; A61K 31/706; A61P 35/00

(52) U.S. Cl. 514/8; 536/27.1; 530/322; 514/52

(57) ABSTRACT

A prodrug composition is provided which includes a pharmaceutical species and an amino acid having a covalent bond to the pharmaceutical species. Particular pharmaceutical species are 6-amino-4-methyl-8-(beta.-D-ribofuranosyl)pyrrolo[4,3,2-de]pyrimido[4,5-c]pyridazine, also known as TCN and by the trade name triciribine; as well as the 5′phosphate of triciribine. TCN and TCNP prodrugs of the present invention have increased bioavailability compared to the parent TCN and TCNP. The inventive prodrug is transported from the gastrointestinal lumen by a specific transporter and is enzymatically cleaved to yield TCN or TCNP, such that TCN or TCNP is delivered to the individual.

### REFERENCE TO RELATED APPLICATIONS

This application is a continuation-in-part of PCT/US2008/058862 filed Mar. 31, 2008, which claims priority of U.S. Provisional Patent Application Ser. No. 60/908,804, filed Mar. 29, 2007. The entire content of these priority applications are hereby incorporated herein by reference.

### FIELD OF THE INVENTION

The present invention generally relates to prodrugs of the nucleoside analog triciribine and its monophosphate, triciribine phosphate, that are designed to enhance oral absorption of the parent drug. Specifically, these prodrugs are the 5′-, 3′-, and 2′-amino acid ester and amino acid phosphoramidate prodrugs of triciribine and triciribine monophosphate. More specifically, these prodrugs are the 5′ amino acid ester and 5′ amino acid phosphoramidate prodrugs of triciribine and triciribine monophosphate.

### BACKGROUND OF THE INVENTION

Triciribine (TCN) and its prodrug, triciribine phosphate (TCNP), were discovered and developed as anticancer compounds by Dr. Leroy Townsend and his collaborators more than three decades ago as detailed in U.S. Pat. No. 4,123,524 and in Schram and Townsend (1971) Tet Lett., 49:4757-60. TCN, while active in cell culture and animal models, proved to be very poorly soluble in aqueous solutions. TCNP, the 5′ monophosphate analog of TCN, was developed to aid in the solubility of the drug. However, neither compound is orally bioavailable. Yang, L., et al., ((2004) Cancer Res, 64(13): 4394-9) reported that TCNP was found to suppress growth and induce apoptosis of cancer cells that overexpress the cellular signaling protein, AKT. Also known as protein kinase B, AKT is a serine/threonine-specific protein kinase, which is a member of the PI3K/AKT/mTOR pathway involved in many key cellular processes that are found to be over-expressed in a variety of cancers. Furthermore, TCNP was shown to directly inhibit phosphorylation and consequent activation of all three isoforms of AKT. These results indicate TCNP selectively inhibits the growth of tumors that overexpress AKT. TCN, TCNP and a variety of analogs have also been tested as antiviral agents, as detailed for example in U.S. Pat. Nos. 5,633,235; 5,827,833; and 6,413,944.

Thus, there exists a need for an orally bioavailable form of TCN and its functional equivalents to broaden to clinical uses of these drugs in the human population.

### SUMMARY OF THE INVENTION

A composition is provided according to embodiments of the present invention including a prodrug having the structural formula:

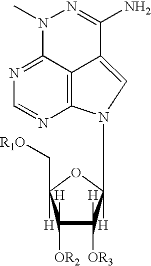

where R₁, R₂, and R₃ are each independently H, or selected from the group consisting of: an amino acid, a dipeptide, a tripeptide, and

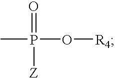

where Z and at least one of R₁, R₂ and R₃ is an amino acid, a dipeptide or a tripeptide;
where R₄ is aliphatic, aryl, or heteroaryl;
or a salt or hydrate thereof.

Optionally, R₁ is selected from the group consisting of: an amino acid, a dipeptide a tripeptide, and

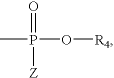

where R₂, and R₃ are each independently H, an amino acid, a dipeptide, a tripeptide or

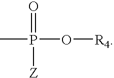

In another option, at least one of R₁, R₂, R₃, and

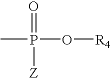

is a substrate for a transporter.

In a further option, the transporter is an intestinal transporter.

In another embodiment of the inventive composition, the prodrug is characterized by at least three-fold greater bioavailability compared to 6-amino-4-methyl-8-(beta.-D-ribofuranosyl)pyrrolo[4,3,2-de]pyrimido[4,5-c]pyridazine, and to 6-amino-4-methyl-8-(beta.-D-ribofuranosyl)pyrrolo[4,3,2-de]pyrimido[4,5-c]pyridazine 5′ phosphate.

In another embodiment of the inventive composition, R₁ is an amino acid or

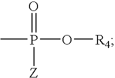

R₂ and R₃ are both H; and Z is an amino acid.

In another embodiment of the inventive composition, at least one of R₁, R₂ and R₃ is: -D-isoleucyl; -L-isoleucyl; -D-valy; -L-valyl; -glycyl; -D-phenylalanyl; -L-phenylalanyl; -D-leucyl; -L-leucyl; -L-aspartyl; -D-alpha-aspartyl; -L-alpha-aspartyl; -D-beta-aspartyl; -L-beta-aspartyl; and -L-prolyl; -D-isoleucyl phosphoramidate; -L-isoleucyl phosphoramidate; -D-valyl phosphoramidate; -L-valyl phosphoramidate; -glycyl phosphoramidate; -D-phenylalanyl phosphoramidate; -L-phenylalanyl phosphoramidate; -D-leucyl phosphoramidate TCN; 5′-O-L-leucyl phosphoramidate TCN; 5′-O-L-aspartyl phosphoramidate; -D-alpha-aspartyl phosphoramidate; -L-alpha-aspartyl phosphoramidate; D-beta-aspartyl phosphoramidate; -L-beta-aspartyl phosphoramidate; or -L-prolyl phosphoramidate.

Described herein in specific embodiments of compositions of the present invention are TCN prodrugs including 5′-O-D-isoleucyl TCN; 5′-O-L-isoleucyl TCN; 5′-O-D-valyl TCN; 5′-O-L-valyl TCN; 5′-O-glycyl TCN; 5′-O-D-phenylalanyl TCN; 5′-O-L-phenylalanyl TCN; 5′-O-D-leucyl TCN; 5′-O-L-leucyl TCN; 5′-O-L-aspartyl TCN; 5′-O-D-alpha-aspartyl TCN; 5′-O-L-alpha-aspartyl TCN; 5′-O-D-beta-aspartyl TCN; 5′-O-L-beta-aspartyl TCN; and TCNP prodrugs including 5′-O-L-prolyl TCN 5′-O-D-isoleucyl phosphoramidate TCN; 5′-O-L-isoleucyl phosphoramidate TCN; 5′-O-D-valyl phosphoramidate TCN; 5′-O-L-valyl phosphoramidate TCN; 5′-O-glycyl phosphoramidate TCN; 5′-O-D-phenylalanyl phosphoramidate TCN; 5′-O-L-phenylalanyl phosphoramidate TCN; 5′-O-D-leucyl phosphoramidate TCN; 5′-O-L-leucyl phosphoramidate TCN; 5′-O-L-aspartyl phosphoramidate TCN; 5′-O-D-alpha-aspartyl phosphoramidate TCN; 5′-O-L-alpha-aspartyl phosphoramidate TCN; 5′-O-D-beta-aspartyl phosphoramidate TCN; 5′-O-L-beta-aspartyl phosphoramidate TCN; and 5′-O-L-prolyl phosphoramidate TCN.

A method of treatment is provided according to embodiments of the present invention which includes administering to a subject in need thereof a therapeutically effective amount of a composition comprising a prodrug having the structural formula:

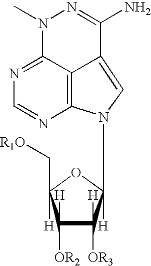

where R₁, R₂ and R₃ are each independently H, or selected from the group consisting of: an amino acid, a dipeptide, a tripeptide, and

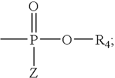

where Z and at least one of R₁, R₂ and R₃ is an amino acid, a dipeptide or a tripeptide;
where R₄ is aliphatic, aryl, or heteroaryl;
and a pharmaceutically acceptable carrier.

In a particular embodiment, the method of treatment includes administration of an inventive composition to a subject having or at risk of having cancer.

A preferred method includes oral administration of an inventive TCN-based prodrug composition.

In another particular embodiment, the subject has a disorder characterized by overexpression of AKT in a tissue of the subject, and the oral administration detectably increases apoptosis in the tissue.

### BRIEF DESCRIPTION OF THE DRAWINGS

FIG. 1 is a stability plot of 5′ O-valyl phosphoramidate TCN, TCN, and TCNP in aqueous buffer;

FIG. 2 is a stability plot of 5′ O-valyl phosphoramidate TCN, TCN, and TCNP in liver homogenates;

FIG. 3 is a stability plot of 5′ O-valyl ester of TCN in buffer;

FIG. 4 is a stability plot of 5′ O-valyl ester of TCN in liver homogenates;

FIG. 5 is a time plot of plasma levels for TCN and TCNP after duodenal administration of 3 mg of TCN (n=3);

FIG. 6 is a time plot of plasma levels for TCN and TCNP after duodenal administration of 3 mg of TCNP (n=3);

FIG. 7 is a time plot of plasma levels for inventive prodrug and conventional TCN and TCNP after duodenal administration of 3 mg of 5′ O-valyl ester prodrug of TCN; and

FIG. 8 is a time plot of plasma levels for inventive prodrug TCN and TCNP after duodenal administration of 3 mg of 5′ valyl phosphoramidates TCN (n=3).

### DETAILED DESCRIPTION OF THE INVENTION

TCN, also referred to as 6-amino-4-methyl-8-(beta.-D-ribofuranosyl)pyrrolo[4,3,2-de]pyrimido[4,5-c]pyridazine, and by the trade name triciribine, is an anti-cancer compound characterized by poor bioavailability and particularly by poor bioavailability when administered orally. For example, TCN is characterized by low lipophilicity and thus shows low intestinal membrane permeability. TCN is poorly soluble in aqueous solutions, such that formulation options for parenteral and peroral administration are limited. TCNP, the 5′ monophosphate of TCN, is also characterized by poor bioavailability.

A TCN-based prodrug according to the present invention has the general form X—Y, where X is the active pharmaceutical species TCN or TCNP to which a promoiety is covalently bonded. The promoiety Y is covalently bound to the active species X. The prodrugs which are the subject of the present invention comprise TCN-based prodrugs for the treatment of cancer. The present invention has utility as a therapeutic agent for the treatment of cancer.

An inventive prodrug enhances the bioavailability of the active species. The prodrugs according to embodiments of the present invention have greater bioavailability than unmodified TCN and TCNP. In particular embodiments, the prodrug TCN-based compositions are characterized by increased transport compared to TCN and TCNP. In this aspect of the invention, transport refers to passive or active transport across the gastrointestinal tract. In other embodiments, the prodrug TCN-based compositions are characterized by increased solubility in aqueous solution compared to TCN.

The promoiety Y is selected to be covalently bindable to the active species X. The promoiety Y includes synthetic and naturally occurring amino acids, di- and polypeptides, pentose sugars, hexose sugars, disaccharides, polysaccharides, C₂-C₂₀ linear or branched alkyl groups, and C₃-C₂₀ alkyl groups having a substituent where the substituent is selected from the group consisting of: amino, hydroxyl, phospho-, phosphatidyl-, and carboxylic groups.

In one embodiment of the present invention, the covalent bond creates a group between the active pharmaceutical species and the promoiety that is selected from the group consisting of: an ester, a phosphoester, phosphoramidate, an amide, an ether, secondary amine, carbamate, and oxime.

The chemical synthesis of an inventive prodrug is appreciated to be largely dictated by the reactive sites available on the active species X or those incorporated therewith, and the corresponding reactive site found on the promoiety Y. For instance, reaction of the 5′ hydroxyl group of TCN, which is a primary hydroxyl group, with a promoiety Y containing a carboxylic group in the presence of a dehydration agent such as N,N′-dicyclohexylcarbodiimide (DCC) yields X—Y in the form of an ester (XCOOY).

While the synthesis of an inventive prodrug is detailed above with chemistry being performed on the active species X in order to form a covalent bond with a subsequent reactant promoiety Y, it is appreciated that modifying chemistry is readily performed on the promoiety Y followed by subsequent reaction with active species X. With respect to following prodrugs of TCNP, for instance, reaction of TCN with an intermediate that contains an activated phosphoryl group linked to the amine group of an amino acid by a phosphoramidate linkage yields a 5′ O-amino acid phosphoramidate triciribine prodrug.

Additionally, it is appreciated that protecting agents are operative herewith to preclude reaction at one or more active sites within an active species X and/or promoiety Y during the course of a coupling reaction. Additionally, a deprotecting agent is operative herein to convert an active species X and/or a promoiety Y into a reactive thiol, amine or hydroxyl substituent. Protecting agents and deprotecting agents are well known in the art. (Theodore W. Green and Peter G. M. Wets, Protective Groups in Organic Synthesis, 2^{nd} Edition (1991)).

Naturally-occurring or non-naturally occurring amino acids are used to prepare the prodrugs of the invention. In particular, standard amino acids suitable as a prodrug moiety include valine, leucine, isoleucine, methionine, phenylalanine, asparagine, glutamic acid, glutamine, histidine, lysine, arginine, aspartic acid, glycine, alanine, serine, threonine, tyrosine, tryptophan, cysteine and proline. Particularly preferred are L-amino acids. Optionally an included amino acid is an alpha-, beta-, or gamma-amino acid. Also, naturally-occurring, non-standard amino acids can be utilized in the compositions and methods of the invention. For example, in addition to the standard naturally occurring amino acids commonly found in proteins, naturally occurring amino acids also illustratively include 4-hydroxyproline, γ-carboxyglutamic acid, selenocysteine, desmosine, 6-N-methyllysine, 3-methylhistidine, O-phosphoserine, 5-hydroxylysine, ε-N-acetyllysine, ω-N-methylarginine, N-acetylserine, γ-aminobutyric acid, citrulline, ornithine, azaserine, homocysteine, β-cyanoalanine and S-adenosylmethionine. Non-naturally occurring amino acids include phenyl glycine, meta-tyrosine, para-amino phenylalanine, 3-(3-pyridyl)-L-alanine, 4-(trifluoromethyl)-D-phenylalanine, and the like.

In one embodiment of an inventive compound, the amino acid covalently coupled to the pharmaceutical species is a non-polar amino acid such as valine, phenylalanine, leucine, isoleucine, glycine, alanine and methionine.

In a further embodiment, more than one amino acid is covalently coupled to the pharmaceutical species. Preferably, a first and second amino acid are each covalently coupled to separate sites on the pharmaceutical species. Optionally, a dipeptide is covalently coupled to the pharmaceutical species. As a further option, a tripeptide is covalently coupled to the pharmaceutical species.

The inventive prodrug enhances the bioavailability of the pharmaceutical species by enhancing its absorption from the gastrointestinal tract while specifically targeting the enzyme responsible for promoiety removal and thus pharmaceutical species release. Bioavailability is defined herein as the amount of the parent drug systemically available in comparison to the total amount of the prodrug delivered to an individual. Bioavailability is typically expressed as % bioavailability and is generally measured by comparing plasma levels of drug after oral administration to plasma levels of drug after intravenous administration. This definition includes first pass metabolism, that is gut and liver metabolism, which when it occurs, occurs before the drug is available systemically. Thus, highly metabolized drugs may be completely absorbed but have a bioavailability less than 100 percent.

It is recognized that TCN and TCNP are characterized by poor bioavailability. Generally, poor oral bioavailability refers to low plasma drug levels after oral administration of active pharmaceutical ingredients at commonly used doses of approximately 0.1-100 mg/kg depending on the compound's potency. An example of a poorly bioavailable (−0.5%) marketed drug is alendronate. Typically, bioavailability of pharmaceutical species decreases with increasing molecular weight. However, in the particular embodiments of the present invention, covalent bonding of a promoiety to the pharmaceutical species X (TCN or TCNP) enhances bioavailability of the pharmaceutical species. In one embodiment, the enhancement is at least 3 fold. In other embodiments, the enhancement is at least 3-, 4-, 5-, 10-, 15-, 20-, 25-, 30-, 35-, 40-, 45-, or 50 fold. The range of 3 to 50 fold is inclusive of all numerical values between 3 and 50 whether whole or decimal, and includes the endpoints. For the enhanced transport, there are a wide variety of known intestinal and cellular transporters that have been identified that could serve as targets for an inventive prodrug. A list of exemplary transporters is given in Table 1. Also in Table 1 is compiled a list of compounds that are known to interact with specific transporters.

TABLE 1
Transporter Targets.

| Transporters | Active species/substrates |
|---|---|
| Amino acid transporters | gabapentin, D-cyclosporin, isobutyl |
| | gaba, L-methyldopa, L-dopa, baclofen |
| Peptide transporter | β-lactam antibiotics, ACE |
| (HPEPT1, HPT1) | inhibitors, valacyclovir, |
| | valganciclovir, cyclosporin, |
| | L-methyldopa, cephalexin |
| Nucleoside transporters | zidovudine, zalcitabine cladribine |
| (CNT1 CNT2, ENT1 ENT2) | ara-C, ara-A, fludarabine, dilazep, |
| | dipyridamole, draflazine hypoxanthine |
| Organic cation transporters | tetraethylammonium, N- |
| (OCT1, ORCTL3) | methylnicotineamide, thiamine, |
| | tyramine, tryptamine, choline, |
| | spermine, spermidine, d-tubocurarine, |
| | procanamide, dobamine, noradrenaline, |
| | serotonin, istamine, corticosterone, |
| | MPP, despramine, qunidine, |
| | verapamil, midazolam |
| Organic anion transporters | methotrexate, cefodizime, ceftriaxone, |
| (MOAT (MRP2), MCT1) | pravastatin, temocaprilat, salicylic |
| | acid, p-amnobenzoic acid, benzoic |
| | acid, nicotinic acid, lactate |
| Glucose transporters | p-nitrophenyl-β-D-glucopyranoside, |
| (GLUT2, GLUT5, SGLT1, | β-D-galactopyranoside |
| SAAT1) | |
| Bile acid transporters | Thyroxine, chlorambucil, crilvastatin |
| (IBAT/ISBT) | |
| Phosphate transporters | fosfomycin, foscarnet, digoxin, |
| (NPT4, NAPI-3B, P-gp) | cyclosporin |
| Vitamin transporters | reduced vitamin C, methotrexate, |
| (SVCT1-2, folate | nicotinic acid, thiamine, vitamin |
| transporters, SMVT) | B-12, R.I.-K(biotin)-Tat9 |

The inventive prodrug is metabolized in the individual to yield the pharmaceutical species and an amino acid. For example, endogenous esterases cleave a described inventive prodrug to yield the pharmaceutical species and amino acid. Table 2 details a nonlimiting list of activation enzymes that are operative to activate various embodiments of prodrugs X—Y by removal of the prodrug moiety Y.

TABLE 2
Activation Enzymes for Inventive Prodrugs.
α/β hydrolase fold family

| | |
|---|---|
| Acylaminoacyl peptidase | Oligopeptidase B |
| (EC 3.4.19.1) | (EC 3.4.21.83) |
| Prolyl oligopeptidase | Biphenyl hydrolase-like |
| (EC 3.4.21.26) | enzyme |
| lecithin: cholesterol | epoxide hydrolase |
| acyltransferase | |
| dipeptidyl peptidase IV (DPP IV) | |

Other peptidases

| | |
|---|---|
| prolidase | prolyl aminopeptidase |
| metalloendopeptidases | tripeptidyl peptidase II |

Alkaline phosphatases and other esterases

| | |
|---|---|
| carboxylesterase | carboxylesterase |
| palmitoyl protein thioesterase | esterase D |
| intestinal alkaline phosphatase | |

Cytochrome p450s

| | |
|---|---|
| cytochrome P450IIA3 (CYP2A3) | cytochrome P(1)-450 |
| cytochrome P450 (CYP2A13) | cytochrome P-450 (P-450 HFLa) |
| cytochrome P450-IIB (hIIB1) | cytochrome P450 4F2 (CYP4F2) |
| cytochrome P4502C9 (CYP2C9) | vitamin D3 25-hydroxylase |
| cytochrome P4502C18 (CYP2C18) | lanosterol 14-demethylase |
| | cytochrome P450 (CYP51) |
| cytochrome P4502C19 (CYP2C19) | cytochrome P450 reductase |
| cytochrome P-450IID | cytochrome P450 PCN3 gene |
| cytochrome P450 monooxygenase | |
| CYP2J2 | |

A composition is provided according to embodiments of the present invention including a TCN-based prodrug having the structural formula:

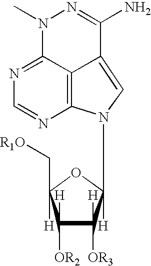

where R₁, R₂ and R₃ are each independently H, or selected from the group consisting of: an amino acid, a dipeptide, a tripeptide, and

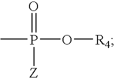

where Z and at least one of R₁, R₂ and R₃ is an amino acid, a dipeptide or a tripeptide; where R₄ is aliphatic, aryl, or heteroaryl.

For the purposes of the invention, “aliphatic” means a straight or branched, saturated cyclic, saturated or unsaturated acyclic hydrocarbon; and includes C₁-C₂₀ alkyl, C₁-C₆ alkyl, C₂-C₄ alkyl, C₃-C₆ cycloalkyl, alkenyl, and alkynyl groups. For the purposes of the invention, “aryl” means a moiety containing an aromatic ring, and includes phenyl, biphenyl, naphthyl, and the like. For the purposes of the invention, “heteroaryl” means a moiety containing an aromatic ring, where the aromatic ring contains at least one nitrogen atom.

Aryl and heteroaryl are optionally substituted with at least one or more moieties selected from alkyl, alkoxy, haloalkyl, and hydroxyl. Preferably, the halogen is fluoro-, chloro, or bromo-.

An included amino acid is an L-amino acid and/or a D-amino acid in particular embodiments of an inventive prodrug. An inventive prodrug is optionally provided as a pharmaceutically acceptable salt or hydrate.

Optionally, R₁ is selected from the group consisting of: an amino acid, a dipeptide a tripeptide, and

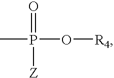

where R₂, and R₃ are each independently H, an amino acid, a dipeptide, a tripeptide or

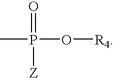

In another embodiment of the inventive composition, R₁ is an amino acid or

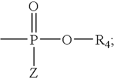

R₂ and R₃ are both H; and Z is an amino acid.

In a particular embodiment of the inventive composition, R₄ is benzyl.

In other embodiment, at least one of R₁, R₂ and R₃ is: -D-isoleucyl; -L-isoleucyl; -D-valy; -L-valyl; -glycyl; -D-phenylalanyl; -L-phenylalanyl; -D-leucyl; -L-leucyl; -L-aspartyl; -D-alpha-aspartyl; -L-alpha-aspartyl; -D-beta-aspartyl; -L-beta-aspartyl; and -L-prolyl; -D-isoleucyl phosphoramidate; -L-isoleucyl phosphoramidate; -D-valyl phosphoramidate; -L-valyl phosphoramidate; -glycyl phosphoramidate; -D-phenylalanyl phosphoramidate; -L-phenylalanyl phosphoramidate; -D-leucyl phosphoramidate TCN; 5′-O-L-leucyl phosphoramidate TCN; 5′-O-L-aspartyl phosphoramidate; -D-alpha-aspartyl phosphoramidate; -L-alpha-aspartyl phosphoramidate; D-beta-aspartyl phosphoramidate; -L-beta-aspartyl phosphoramidate; or -L-prolyl phosphoramidate.

Particular examples of TCN-based prodrugs according to the present invention include TCN-linked to a non-polar amino acid through an ester linkage; and TCN linked to a non-polar amino acid through a phosphoramidate linkage.

Specific TCN prodrugs of the present invention include 5′-O-D-isoleucyl TCN; O-L-isoleucyl TCN; 5′-O-D-valyl TCN; 5′-O-L-valyl TCN; 5′-O-glycyl TCN; 5′-O-D-phenylalanyl TCN; 5′-O-L-phenylalanyl TCN; 5′-O-D-leucyl TCN; TCN; 5′-O-L-aspartyl TCN; 5′-O-D-alpha-aspartyl TCN; 5′-O-L-alpha-aspartyl TCN; 5′-O-D-beta-aspartyl TCN; 5′-O-L-beta-aspartyl TCN; and 5′-O-L-prolyl TCN; as well as 5′-O-D-isoleucyl phosphoramidate TCN; phosphoramidate TCN; 5′-O-D-valyl phosphoramidate TCN; 5′-O-L-valyl phosphoramidate TCN; 5′-O-glycyl phosphoramidate TCN; 5′-O-D-phenylalanyl phosphoramidate TCN; 5′-O-L-phenylalanyl phosphoramidate TCN; phosphoramidate TCN; 5′-O-L-leucyl phosphoramidate TCN; 5′-O-L-aspartyl phosphoramidate TCN; 5′-O-D-alpha-aspartyl phosphoramidate TCN; 5′-O-L-alpha-aspartyl phosphoramidate TCN; 5′-O-D-beta-aspartyl phosphoramidate TCN; 5′-O-L-beta-aspartyl phosphoramidate TCN; and 5′-O-L-prolyl phosphoramidate TCN.

Examples of a dipeptide TCN-based prodrug include 5′-O-L-alagly TCN and 5′-O-L-alagly phosphoramidate TCN.

In other embodiments of the present invention, the amino acid, dipeptide or tripeptide substrate for a transporter is a substrate for an intestinal transporter. In more particular embodiments, an amino acid included in an inventive prodrug includes an amino acid which is a substrate for a nutrient transporter such as PEPT1 and/or HPT1.

The compositions and methods of the invention comprehend combinations of TCN-based prodrugs of the invention with any other known antiproliferative and/or anticancer agents; including one or more compounds and compositions referred to in United States Patent Application Publications US 2006/0247188 and US 2006/0030536 including inhibitors of Jak2/Stat3 signaling pathway, inhibitors of the P13k/Akt pathway, and inhibitors of AKt; the contents of which publications are hereby incorporated herein by reference. In another embodiment of the present invention, the composition further comprises at least one agent that is an anti-neoplastic compound.

In another embodiment, the anti-neoplastic compound is selected from floxuridine, gemcitabine, cladribine, dacarbazine, melphalan, mercaptopurine, thioguanine, cis-platin, and cytarabine.

In other embodiments of the present inventive compositions, and of the antiproliferative methods described herein, the TCN-based prodrug is in combination with least one chemotherapeutic agent selected from: 13-cis-Retinoic Acid, 2-Amino-6-, Mercaptopurine, 2-CdA, 2-Chlorodeoxyadenosine, 5-fluorouracil, 5-FU, 6-TG, 6-Thioguanine, 6-Mercaptopurine, 6-MP, Accutane, Actinomycin-D, Acyclovir, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anadron, Anastrozole, Arabinosylcytosine, Ara-C, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, hydrochloride, Daunorubicin liposomal, DaunoXome, Decadron, Delta-Cortef, Deltasone, Denileukin diftitox, DepoCyt, Dexamethasone, Dexamethasone acetate, dexamethasone sodium, phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, Hydrochlorine, Medralone, Medrol, Megace, Megestrol, Megestrol Acetate, Melphalan, Mercaptopurine, Mesna, Mesnex, Methotrexate, Methotrexate Sodium, tacrine Methylprednisolone, Mylocel, Letrozole, paclitaxel, taxol, vinblastin, and vincristine.

The prodrugs according to the present invention are effective in treating one or more varieties of cancer. In one embodiment, the prodrugs according to the present invention are effective in treating one or more varieties of cancer characterized by overexpression of AKT. AKT, also known as protein kinase B is normally activated by a variety of growth and angiogenic factors and cytokine receptors which in turn activate phosphatidylinositol-3-kinase (PI3K). Upon activation, PI3K phosphorylates phosphotidylinositol 4,5-bisphosphate (PIP2) to phosphatidylinositol (3,4,5)-triphosphate (PIP3). AKT is then activated by phosphorylation on Thr 308 and Ser 473 (AKT1), 474 (AKT2) or 472 (AKT3). Following its activation, AKT phosphorylates its substrates which induce transcription, translation, cell cycle progression, glucose metabolism and inhibition of apoptosis. AKT's best-known target is the mammalian target of Rapamycin (mTOR) which induces cell proliferation, growth, and angiogenesis. AKT also inhibits apoptosis by phosphorylating and thus inhibiting several proapoptotic proteins such as Bc1-2-associated death promoter (BAD) protein and the forkhead transcription factor, FoxO. Furthermore, AKT activates NF-KB which then activates several anti-apoptotic proteins [3]. Since the PI3K/AKT/mTOR pathway induces the major processes which are upregulated in tumorigenesis, this pathway is a good target for inhibition in many cases of cancer.

AKT is involved in malignant transformation. AKT has three isoforms: AKT1, AKT2 and AKT3 and each of them have a significant role in several stages of malignant transformation. Many human cancers contain hyperactivated AKT. This can be due to amplification and/or overexpression of AKT itself as well as genetic alterations upstream of AKT including overexpression of receptor tyrosine kinases and/or their ligands, and mutation or deletion of the phosphatase PTEN. Activation of the PI3K/AKT/mTOR pathway is an early event in carcinogenesis, which raises the threshold for apoptosis of damaged cells. Proof-of-concept of the involvement of AKT in oncogenesis has been demonstrated preclinically by showing that ectopic expression of AKT induces malignant transformation and promotes cell survival while disruption of AKT pathways inhibits cell growth and induces apoptosis. More importantly, over expression of receptor tyrosine kinases such as EGFR and their ligands such as IGF-1, AKT overexpression, and/or loss of PTEN, all of which result in hyperactivation of AKT, are associated with poor prognosis, resistance to chemotherapy, and shortened survival time of cancer patients. Activated AKT has been found in leukemia (70% of patients), colorectal (57% of patients), ovarian (36% of patients), pancreatic (32% of patients), and breast (28% of patients) cancers. PI3K and PTEN have also been implicated with brain, bladder and endometrial cancers. Many transforming events that do not result in direct genetic modification of PI3K, AKT or PTEN can still cause activation of the PI3K/AKT/PTEN pathway. Three examples of such events are the BCR/ABL translocation which is the causative event in chronic myelogenous leukemia, amplification of HER-2/neu seen frequently in primary breast carcinomas, and amplification of the epidermal growth factor receptor (EGFR) seen in multiple carcinomas. Inhibition of the PI3K/AKT pathway in these tumors in-vitro significantly enhances their responsiveness to chemotherapeutic treatment. See Testa, J. R. and A. Bellacosa (2001), Proc Natl Acad Sci USA, 98(20):10983-5; Testa, J. R. and P. N. Tsichlis, (2005) Oncogene, 24(50): 7391-3; Paez, J. and W. Sellers, (2003) PI3K/PTEN/Akt Pathway: A Critical Mediator of Oncogenic Signaling, in Signal Transduction in Cancer, D. Frank, Editor. 2003, Kluwer Academic Publishers Netherlands; Datta, S. R., et al. (1999) Cellular survival: a play in three Akts. Genes Dev, 13(22): 2905-27; Fayard, E. et al. (2005) J Cell Sci, 118 (Pt 24: 5675-8); Nicholson, K. M. and N. G. Anderson, (2002) Cell Signal, 2002, 14(5): p. 381-95; Mirza, A. M., Fayard, E. et al. (2000) 2000. 11(6: 279-92; Cheng, J. and S. Nicosia, (2001) AKT signal transduction pathway in oncogenesis, in Encyclopedic Reference of Cancer, D. Schwab, Editor. 2001, Springer: Berlin, Germany, p. 35-7.

The prodrugs according to the present invention are effective in treating one or more varieties of cancer; including but not limited to the varieties stated herein. In one embodiment, the inventive prodrug is formulated for administration to a human individual, and bioavailability and fraction absorbed measurements refer to measurements made in humans. However, it is appreciated that the inventive prodrug and method of treatment may be indicated in non-human applications as well. Thus, the inventive prodrug is advantageously administered to a non-human organism such as a rodent, bovine, equine, avian, canine, feline or other such species wherein the organism possesses an enzyme and a membrane transporter for which the prodrug is a substrate.

A method of treatment according to the present invention basically consists of administering a therapeutically effective amount of an inventive prodrug to an organism. It is recognized that the methods of treatment of the invention have commercial value. Thus, the inventive methods have application in the medical and pharmaceutical industries. Variable dosing regimens are operative in the method of treatment. While single dose treatment is effective in producing therapeutic effects, it is noted that longer courses of treatment such as several days to weeks have previously been shown to be efficacious in prodrug therapy (Beck et al., Human Gene Therapy, 6:1525-30 (1995)). While dosimetry for a given inventive prodrug will vary, dosimetry will depend on factors illustratively including target cell mass, effective active species X cellular concentration, efficiency of transport, systemic prodrug degradation kinetics, and secondary enzymatic cleavage that reduces active species lifetime.

A prodrug is administered by a route determined to be appropriate for a particular subject by one skilled in the art. For example, a prodrug is administered orally; parentally, such as intravenously; by intramuscular injection; by intraperitoneal injection; intratumorally; transdermally; rectally. The exact dose of prodrug required is appreciated to vary from subject to subject, depending on the age, weight and general condition of the subject, the severity of the diseases being treated, the particular active species, the mode of administration, and the like. An appropriate dose is readily determined by one of ordinary skill in the art using only routine experimentation given the teachings herein. Generally, dosage is in the range of about 0.5-500 mg per m².

In a particularly preferred embodiment of the present invention, the inventive TCN-based prodrug is administered orally. In another embodiment, a parenteral administration of TCN and/or TCNP, including intravenous administration, is followed by oral administration of the inventive TCN-based prodrug. It is recognized that such parenteral administration followed by oral dosing of the TCN-based prodrug can be done for the purpose of maintenance dosing.

Depending on the intended mode of administration, the substrate can be in pharmaceutical compositions in the form of solid, semi-solid or liquid dosage forms, such as, for example, tablets, suppositories, pills, capsules, powders, liquids, or suspensions, preferably in unit dosage form suitable for single administration of a precise dosage. Time release preparations are specifically contemplated as effective dosage formulations. The compositions will include an effective amount of the selected substrate in combination with a pharmaceutically acceptable carrier and, in addition, may include other medicinal agents, pharmaceutical agents, carriers, or diluents.

For solid compositions, conventional nontoxic solid carriers include, for example, pharmaceutical grades of mannitol, lactose, starch, magnesium stearate, sodium saccharine, talc, cellulose, glucose, sucrose and magnesium carbonate. Liquid pharmaceutically administrable compositions can, for example, be prepared by dissolving or dispersing an active compound with optimal pharmaceutical adjuvants in an excipient, such as water, saline, aqueous dextrose, glycerol, or ethanol, to thereby form a solution or suspension. If desired, the pharmaceutical composition to be administered may also contain minor amounts of nontoxic auxiliary substances such as wetting or emulsifying agents, pH buffering agents, for example, sodium acetate or triethanolamine oleate. Actual methods of preparing such dosage forms are known, or will be apparent, to those skilled in this art; for example, see Remington's The Science and Practice of Pharmacy (20^{th} Edition).

For oral administration, fine powders or granules may contain diluting, dispersing, and/or surface active agents, and may be presented in water or in a syrup, in capsules or sachets in the dry state or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated.

Parenteral administration is generally by injection. Injectables can be prepared in conventional forms, either liquid solutions or suspensions, solid forms suitable for solution or prior to injection, or as suspension in liquid prior to injection or as emulsions.

TCN-based prodrugs according to the present invention are readily created to treat a variety of proliferative disorders, including cancer. In particular embodiments, the TCN-based prodrug is administered to a subject in need thereof as an antiproliferative agent, for example, to treat cancer such as a tumor or other pathological cell proliferative disorder. The antiproliferative and/or anticancer activity of an inventive prodrug of the invention, as well as its parent drug, is determinable by methods known to the ordinarily skilled artisan and include cell-based growth inhibition-, clonogenic-, and cytotoxic assays. For example, see Hoffman R M, (1991) J. Clin. Lab. Anal. 5(2), 133-43; Franken et al. (2006) Nature Protocols 1: 2315-2319; and Shoemaker R H (2006), Nat. Rev. Cancer, 6(10), 813-23. See also, United States patent application publications 2006/0030536 and 2006/0247188; the contents of which publications are hereby incorporated herein by reference.

In one embodiment, a method of treatment according to the present invention includes administering to a subject in need thereof a therapeutically effective amount of an inventive TCN-based prodrug to a subject that has or is at risk of having cancer. For the purposes of the invention, cancer includes leukemia, lymphoma, carcinoma, AIDS-related-, skin-, anal-, appendix-, bladder-, brain-, breast-, colon-, cervical-, testicular-, colorectal-, and prostate cancer.

A method of treatment according to the present invention includes administering a therapeutically effective amount of an inventive TCN-based prodrug to an organism possessing an enzyme and a membrane transporter wherein the TCN-based prodrug is a substrate for both.

In a particular embodiment of the inventive method for delivering TCN to an individual the method includes the step of administering an inventive TCN-based prodrug as described herein to the gastrointestinal lumen of an individual subject. The TCN-based prodrug is transported from the gastrointestinal lumen by a specific transporter and enzymatically cleaved to yield TCN, thereby delivering TCN to the individual subject.

An antiproliferative method is provided according to embodiments of the present invention which includes administering to a subject in need thereof a therapeutically effective amount of a composition comprising an TCN-based prodrug having the structural formula:

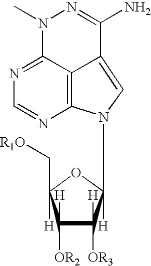

where R₁, R₇ and R₃ are each independently H, or selected from the group consisting of: an amino acid, a dipeptide, a tripeptide, and

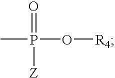

where Z and at least one of R₁, R₂ and R₃ is an amino acid, a dipeptide or a tripeptide; where R₄ is aliphatic, aryl, or heteroaryl; and a pharmaceutically acceptable carrier.

A preferred antiproliferative method includes oral administration of an inventive TCN prodrug composition to a subject. In particular embodiments, a method of treating a subject includes administration of a prodrug compound selected from 5′-O-D-isoleucyl TCN; 5′-O-L-isoleucyl TCN; 5′-O-D-valyl TCN; 5′-O-L-valyl TCN; 5′-O-glycyl TCN; 5′-O-D-phenylalanyl TCN; 5′-O-L-phenylalanyl TCN; 5′-O-D-leucyl TCN; 5′-O-L-leucyl TCN; 5′-O-L-aspartyl TCN; 5′-O-D-alpha-aspartyl TCN; 5′-O-L-alpha-aspartyl TCN; 5′-O-D-beta-aspartyl TCN; 5′-O-L-beta-aspartyl TCN; and 5′-O-L-prolyl TCN; as well as 5′-O-D-isoleucyl phosphoramidate TCN; 5′-O-L-isoleucyl phosphoramidate TCN; 5′-O-D-valyl phosphoramidate TCN; 5′-O-L-valyl phosphoramidate TCN; 5′-O-glycyl phosphoramidate TCN; 5′-O-D-phenylalanyl phosphoramidate TCN; 5′-O-L-phenylalanyl phosphoramidate TCN; 5′-O-D-leucyl phosphoramidate TCN; 5′-O-L-leucyl phosphoramidate TCN; 5′-O-L-aspartyl phosphoramidate TCN; 5′-O-D-alpha-aspartyl phosphoramidate TCN; 5′-O-L-alpha-aspartyl phosphoramidate TCN; 5′-O-D-beta-aspartyl phosphoramidate TCN; 5′-O-L-beta-aspartyl phosphoramidate TCN; and 5′-O-L-prolyl phosphoramidate TCN; and a combination of any of these.

In another embodiment of the antiproliferative method of the present invention, the administered composition further comprises an anti anti-neoplastic compound selected from floxuridine, gemcitabine, cladribine, decarbazine, melphalan, mercaptopurine, thioguanine, cis-platin, and cytarabine. In another embodiment, the administered composition further comprises a chemotherapeutic agent described herein.

In another embodiment of the antiproliferative method of the present invention, the method further comprises administering an antiproliferative or chemotherapeutic agent described herein, to a subject in need thereof.

In another particular embodiment of the antiproliferative method, the subject has a disorder characterized by overexpression of AKT in a tissue of the subject, and oral administration of the inventive TCN-based prodrug detectably increases apoptosis in the tissue.

In another embodiment, the tissue is a tumor.

In a more particular embodiment, the tumor is malignant.

Methods of synthesizing a TCN-based prodrug are provided herein. Broadly described, embodiments of a method of synthesizing a TCN-based prodrug include protecting one or more reactive groups of TCN or TCNP, conjugating a promoiety to TCN or TCNP, and deprotecting the protected reactive group or groups, resulting in a promoiety-TCN- or promoiety-TCNP conjugate prodrug.

Exemplary methods of synthesizing TCN-based prodrugs are described in detail in Examples herein.

The TCN-based prodrug according to the present invention is provided as the free base in particular embodiments. Optionally, the TCN-based prodrug according to the present invention is provided as a pharmaceutically acceptable salt. For example, an inventive TCN-based prodrug is illustratively provided as a salt of an inorganic or organic acid such as hydrochloric acid, sulfuric acid, phosphoric acid, acetic acid, citric acid, benzoic acid, maleic acid, fumaric acid, succinic acid, methanesulfonic acid, p-toluenesulfonic acid or trifluoroacetic acid. Further optionally, the TCN prodrug is provided as a hydrate.

The examples presented below are intended to illustrate a particular embodiment of the invention and are not intended to limit the scope of the specification, including the claims, in any way.

### Example 1

### Synthesis of Amino Acid Prodrugs of TCN that Contain No Nucleophile on the Amino Acid Side Chain

Abbreviations used herein: TCN is 6-amino-4-methyl-8-(beta.-D-ribofuranosyl)pyrrolo[4,3,2-de]pyrimido[4,5-c]pyridazine, TCNP is 6-amino-4-methyl-8-(beta.-D-ribofuranosyl)pyrrolo[4,3,2-de]pyrimido[4,5-c]pyridazine 5′ phosphate, DCC is N,N′-Dicyclohexylcarbodiimide, DMF is dimethylformamide, equ is equivalents, HPLC is High Performance Liquid Chromatography, NMR is Nuclear Magnetic Resonance, and TFA is Trifluoroacetic acid, DCM is dichloromethane, DMAP is dimethyl amine pyridine, TBAF is Tetrabutylammonium fluoride, t-Boc is tertiary-butyloxycarbonyl, Et₃N is triethylamine, NMI is N-methylimidazole, THF is tetrahydrofuran, and t-butyl is tertiary butyl.

As shown in Scheme 3, DCC (1.5 equ) in DMF solution is added to an anhydrous DMF solution containing TCN (1 equ), dried N Boc protected Amino acid (1.5 equ) and dried dimethyl amine pyridine (1.5 equ). The mixture is stirred at room temperature for 20 hours, during which time a white precipitate forms. At the end of this period, 5 mL of anhydrous ethanol is added and the reaction mixture is stirred for an additional 1 hour. The solution is filtered to remove the precipitate and the filtrate is evaporated to dryness. The residue is subjected to flash silica gel chromatography to separate the 2′ and 3′ monoester and diester prodrugs from the 5′ monoester prodrug. The protected prodrug is collected and dried by vacuum. The residue is deprotected by 50% TFA in dichloromethane and purified by preparative HPLC. The prodrug compounds which are made by this method are characterized by H-NMR, C13-NMR, Mass Spectrometry, and elemental analysis. The purities of the compounds are verified by HPLC and elemental analysis and the % purity is determined. All of the compounds are isolated as TFA salts and tested for solubility in water.

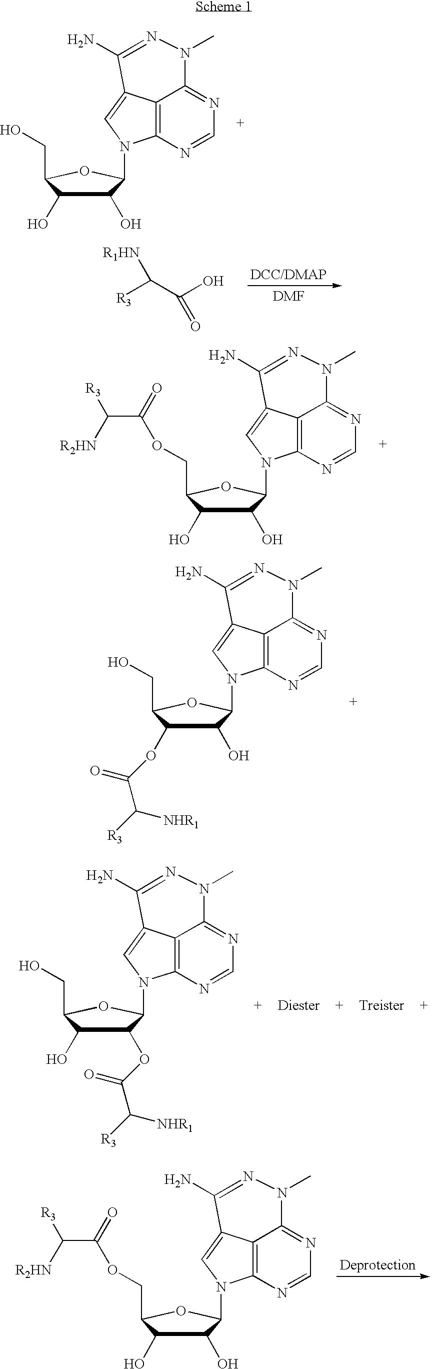

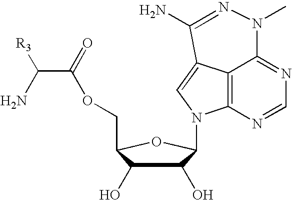

Scheme I shows synthesis of TCN amino acid prodrugs that do not contain a nucleophile in the amino acid side chain. In this scheme R₁ represents a protective group such as t-Boc that is commonly used to protect amine groups and R₃ is the side chain of the amino acid used in the synthesis of the prodrug.

### Example 2

Synthesis of 5′ O-valyl triciribine [6-Amino-4-methyl-8-[5-valyl-(β-D-ribofuranosyl)-pyrrolo[4,3,2-de]]pyrimido[4,5-c]pyridazine]. As illustrated in Scheme 2, triciribine (1 equ), N-Boc valine (1.2 equ), and DMAP (1.2 equ) are dissolved in anhydrous DMF and DCC (1.2 equ) in anhydrous DMF is added dropwise at room temperature. The reaction mixture is stirred for 20 hours, after which the solvent is evaporated at 42° C. under high vacuum until dryness (1). The Boc protection group is removed by treating with Trifluoroacetic acid in DCM for 4 hours. The valyl ester is purified using flash silica gel chromatography with 9:1 DCM to MeOH as eluent and preparative HPLC to obtain the pure product (2). The structure is confirmed by H¹ NMR and LC/MS/MS.

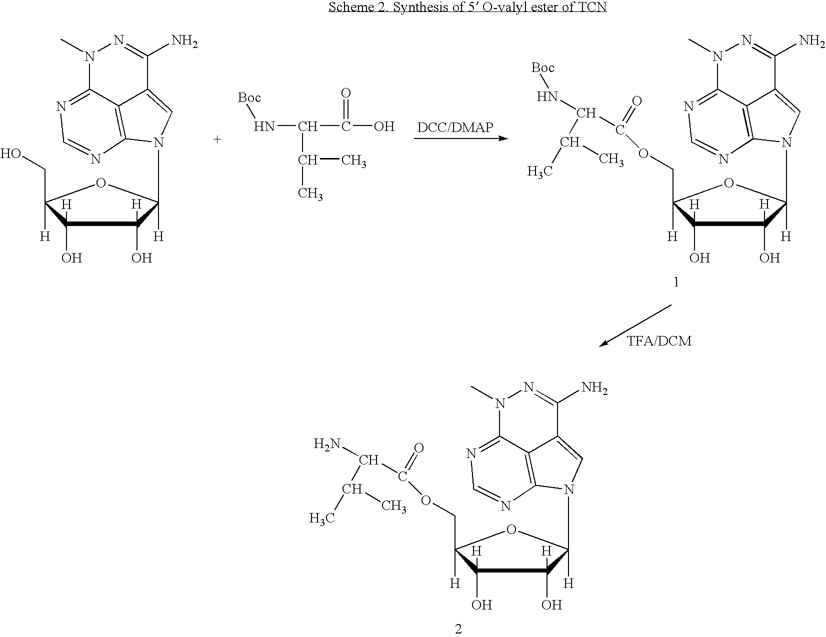

### Example 3

Synthesis of 5′ O-valyl phosphoramidate triciribine [6-Amino-4-methyl-8-[5-[(Methylphenylphosphoryl)P→N-L-valylate]-(β-D-ribofuranosyl)pyrrolo[4,3,2-de]]pyrimido[4,5-c]pyridazine]. As illustrated in Scheme 3, phenyl-dichlorophosphate (1 equ) and valine methyl ester hydrochloride (1 equ) are combined in anhydrous dichloromethane. Triethylamine (2 equ) in anhydrous dichloromethane is added to the mixture at −78° C. over the course of 2 hours. The reaction mixture is allowed to come to room temperature and incubated and additional 20 hours. After this incubation, the solvent is evaporated and anhydrous diethyl ether is added to the residue. The resulting mixture is filtered under argon and the filtrate is dried to obtain (3). 1 (5 equ) is dissolved in anhydrous THF and combined with N-methyl imidazole (5 equ), which is then added to a solution of triciribine (1 equ) in THF at −78° C. The reaction mixture is allowed to come to room temperature and is stirred an additional 20 hours. The crude phosphoramidate prodrug (4) is purified using flash silica gel chromatography with 9:1 DCM to MeOH as eluent and preparative HPLC to obtain the pure product.

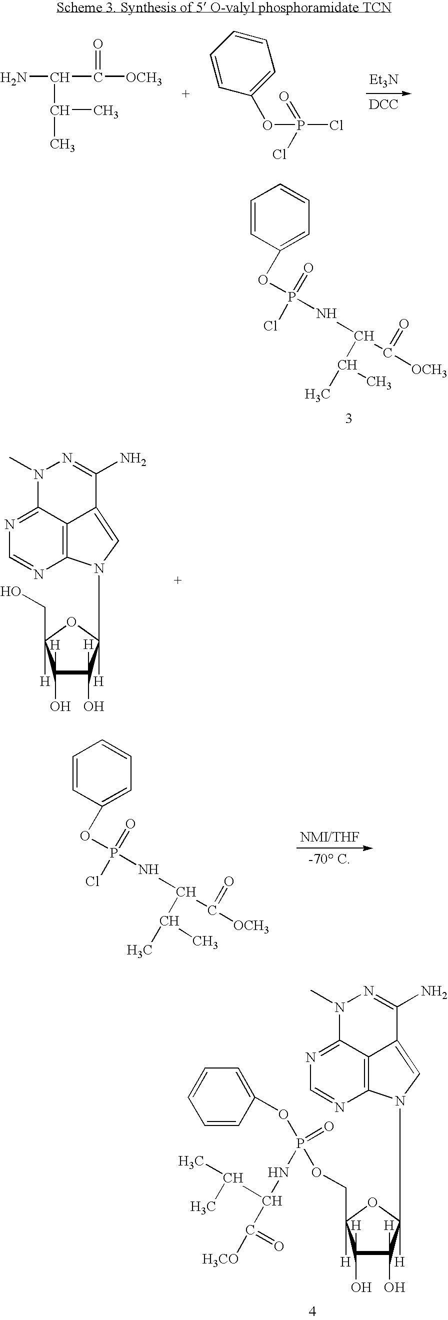

### Example 4

### Synthesis of the Amino Acid TCN-Based Prodrugs with Amino Acids Containing a Nucleophilic Side Chain, and General Procedure for Selectively Protecting 2′- and 3′-hydroxyl groups of triciribine

For this method, the 2′ and 3′ hydroxyl groups of TCN are protected to prevent formation of the 2′ and 3′ mono and diester prodrugs, see Scheme 4. Tert-butyldimethylchlorosilane (1.2 equ) in 20 ml DMF is added to a solution of dried TCN (3 equ) in 10 mL of dry N,-N-dimethylformamide (DMF) and the mixture is stirred under argon at room temperature for 24 h. The solvent is removed in vacuo at 42° C. and the residue is purified by silica flash chromatography with a solvent of 92:8 dichloromethane and methanol. The product, 5′-O-(tert-butyldimethylsilyl)-[6-amino-4-methyl-8-[(β-D-ribofuranosyl)pyrrolo[4,3,2-de]pyrimido[4,5-C]pyridazine] (5), is crystallized from a mixture of hot chloroform and hexane, and the yield determined.

DCC (9 equ) is added to a solution of laevulin acid (9 equ) in dry ethyl acetate (25 mL) and the mixture is stirred under the protection of argon room temperature for 1.5 h. The mixture is filtered under argon and added to a suspension of 16 (1 equ) and N,N-dimethylaminopyridine (1.2 equ) in dry ethyl acetate (25 mL). The reaction mixture is stirred at room temperature for 4 h, dry EtOH (10 mL) is added and stirred an additional 30 min, and the reaction is filtered. The filtrate is extracted three times with 50 mL of ice-cold saturated NaHCO solution and the combined aqueous solution is back washed three times with 50 mL ethyl acetate. The combined ethyl acetate solution is extracted with ice-cold water (3×50 mL) and the combined water layer is back washed three times with ethyl acetate. The washed ethyl acetate solution is evaporated to dryness. The residue is subjected to flash column silica gel chromatography developed with dichloromethane followed by 94:6 dichloromethane and methanol. The product (6) is isolated, and the yield determined.

The syrup of 17 (1 equ) is dissolved in a solution of 1 M TBAF in THF (5 ml) containing glacial acetic acid (0.5 mL). The reaction mixture is stirred at room temperature for 2 h and is then evaporated to dryness in vacuo in the presence of toluene. The residue is separated by silica gel flash chromatography developed with 95:5 and 92:8 dichloromethane and methanol, yielding a clear gum, which is further separated by silica gel flash chromatography developed with 9:1 dichloromethane and methanol. The dried product (7) is isolated, and the yield determined.

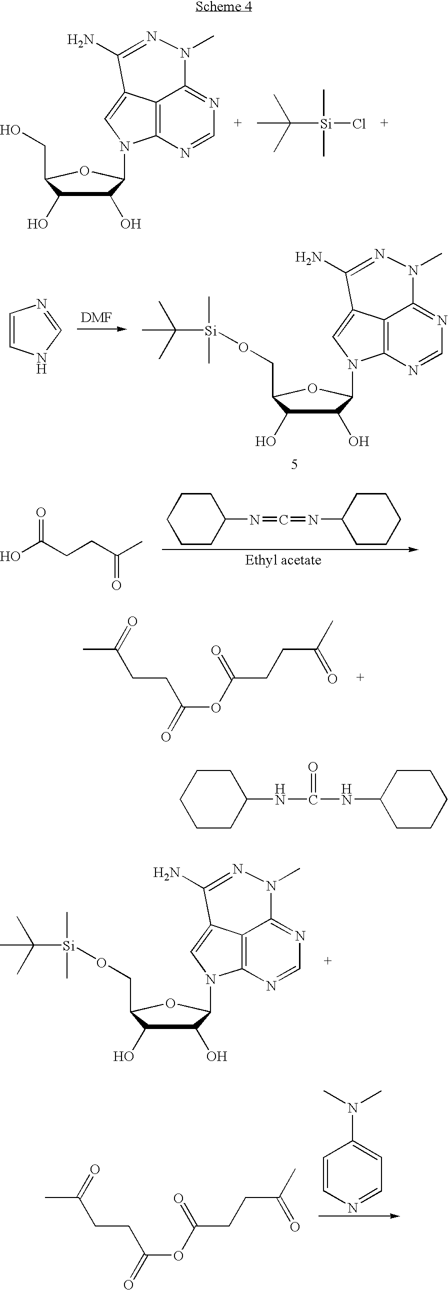

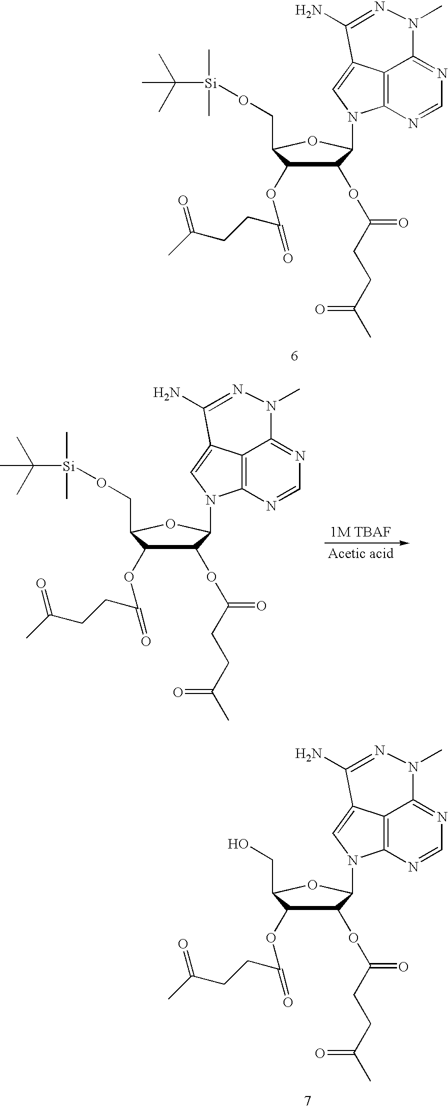

Scheme 4 shows the synthesis of 2′,3′-O-dilevulinate-[6-amino-4-methyl-8-(β-D-ribofuranosyl)pyrrolo[4,3,2-de]pyrimido[4,5-C]pyridazine] (7), which can be used as an intermediate for the synthesis of 5′ O amino acid ester prodrugs of TCN in which the amino acid has a reactive side chain group.

The synthesis of 5′ O-ester TCN prodrugs is shown in Scheme 5. DCC solution (1.5 equ) in DMF is added over a time course of 10 minutes to a DMF solution containing the protected TCN (7) is isolated, and the yield determined. (1 equ), N protected Amino acid (1.5 equ) and Dimethyl amine pyridine (1.5 equ). The mixture is stirred at room temperature for 20 hours during which time a white precipitate formed. At the end of this period, 5 mL of anhydrous ethanol is added and the reaction mixture is stirred for an additional 1 hour. The solution is filtered to remove the precipitate and the filtrate is evaporated to dryness. The residue, containing the protected TCN prodrug (8) is dissolved in ethyl acetate, and consecutively washed with saturated ammonium chloride aqueous solution (1 time) and water (1 time). The organic phase is dried in vacuo and the residue is subjected to flash silica gel chromatography. The product, (8), is dissolved in 4:1 pyridine and glacial acetic acid followed by addition of 0.1 M of hydrazine monohydrate in 4:1 pyridine and glacial acetic acid to remove the levulinic groups from the 2′ and 3′ OH groups of the TCN. The deprotected prodrug, (9), is separated by flash silica gel chromatography and the amino acid protection group(s) are removed either by TFA (t-Boc, t-butyl) or by hydrogenolysis (Z or benzyl groups) or both and the final product, 21, is purified by preparative HPLC. All of the compounds are identified by H-NMR, C13-NMR, Mass Spectrometry, and elemental analysis. The purities of the compounds are verified by HPLC and elemental analysis and the % purities are determined. All of the compounds are isolated as TFA, and tested for solubility in water.

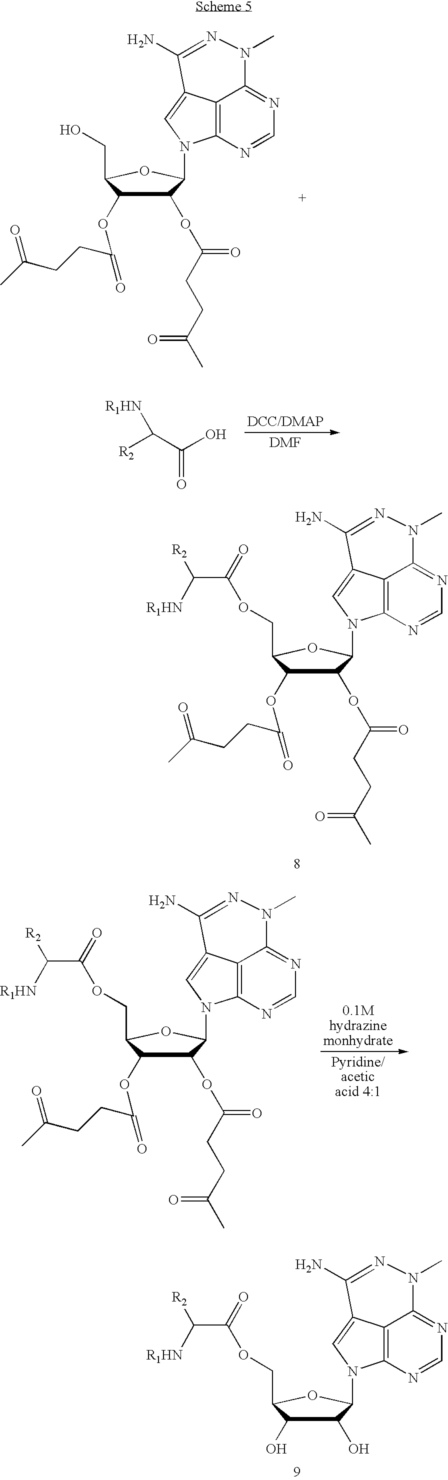

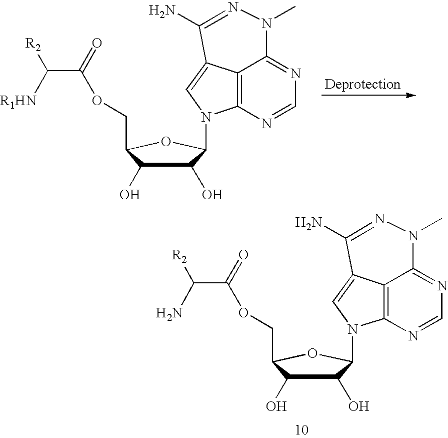

Scheme 5 shows synthesis of 5′ O-ester TCN prodrugs (10) using an amino acid that can have a reactive side chain group. Such amino illustratively include lysine, serine, cysteine, glutamine, asparagine, threonine, tyrosine and an amino acid having the formula HOOC—(CH₂)—CH(NH₂)—COOH where n is an integer in the range of 1-6, inclusive. In this scheme R₁ represents a protective group such as t-Boc that is commonly used to protect amine groups, represents a protected side chain of the amino acid that is used in the synthesis of the prodrug and R₃ is the deprotected side chain of the amino acid used in the synthesis of the prodrug.

### Example 5

### Synthesis of 5′ O-ester TCN Prodrugs Including an Amino Acid Containing a Reactive Carboxylic Group of the Side Chain

In particular embodiments, an amino acid conjugated to TCN has the formula HOOC—(CH₂)—CH(NH₂)—COOH where n is an integer in the range of 1-6, inclusive.

Scheme 6 illustrates synthesis of 5′ O-ester TCN prodrugs (13) in which the reactive carboxylic group of the side chain of the amino acid, aspartic acid (n=1), glutamic acid (n=2) or analogs thereof (n=3-6), is linked to the 5′ hydroxyl group of TCN.

Protection of 2′ and 3′ OH groups of TCN is performed as described in Example 4 and Scheme 4. The synthesis of 5′ O-ester TCN prodrugs (Scheme 6) is as described in Example 4, but uses a starting protected amino acid that is blocked at the alpha carboxylic group and the alpha amino group and with a carboxylic group on the side chain of the amino acid.

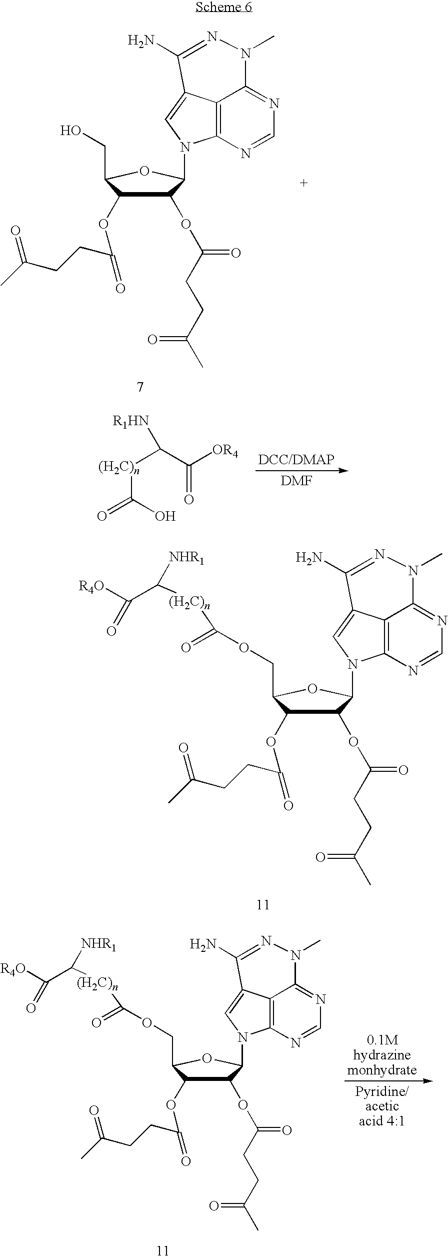

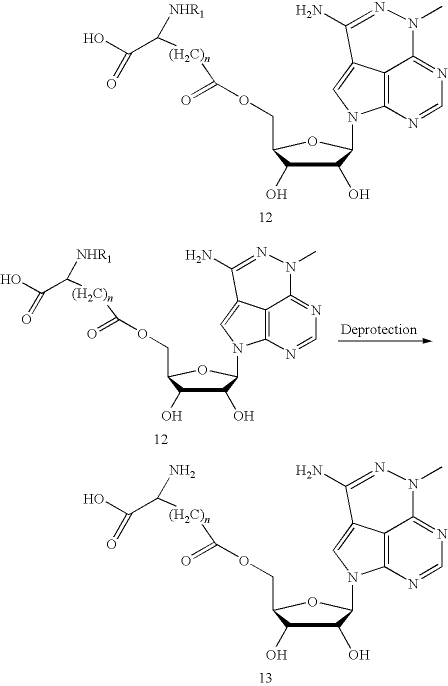

Scheme 6 shows synthesis of 5′ O— ester TCN prodrugs (13) in which the reactive carboxylic group of the side chain of amino acids aspartic acid (n=1), glutamic acid (n=2) and their analogs (n=3-6) is linked to the 5′ hydroxyl group of TCN. In this scheme R₁ represents an amine protective group such as t-Boc commonly used to protect amine groups and R₄ is a protective group, such as a benzyl group, commonly used to protect carboxylic groups.

### Example 6

### General Procedures for Selectively Protecting 3′ and 5′-Hydroxyl Groups of Triciribine Scheme 7

With reference to the detailed description illustrated in Example 4, scheme 7 sets the forth general procedures for selectively protecting 3′ and 5′-hydroxyl groups of triciribine

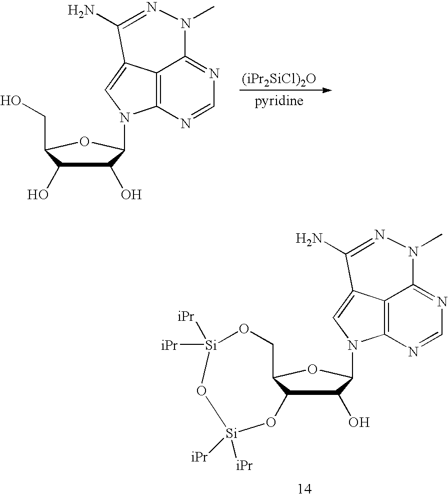

Triciribine (TCN) was treated with 1,3-Dichloro-1,1,3,3-tetraisopropyldisiloxane in the presence of imidazole to obtain 3′,5′-O-Tetraisopropyldisiloxane TCN (14).

### Example 7

### General Procedures for Selectively Protecting 3′ and 5′-Hydroxyl Groups of Triciribine

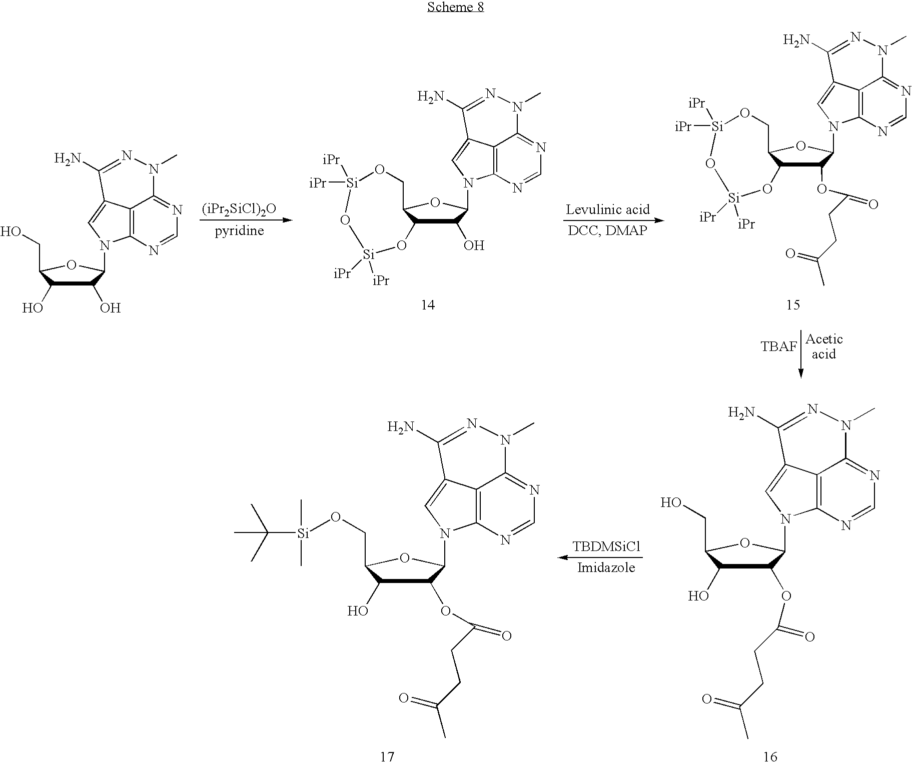

With reference to the detailed description illustrated in Example 4, scheme 8 sets forth the General procedures for selectively protecting 3′ and 5′-hydroxyl groups of triciribine.

Triciribine (TCN) was treated with 1,3-Dichloro-1,1,3,3-tetraisopropyldisiloxane in the presence of imidazole to obtain 3′,5′-O-Tetraisopropyldisiloxane TCN (14). The 2′-hydroxyl group was then protected as levulinate ester (15). The tetraisopropyldisiloxane group was removed by tetrabutylammonium fluoride in the presence of acetic acid to obtain (16) followed by protection of 5′ hydroxyl group with TBDMS group to obtain 2′-O-lev-5′-O-TBDMS-TCN or 2′-O-levulinate-5′-O-(tert-butyldimethylsilyl)-[6-amino-4-methyl-8-(β-D-ribofuranosyl)pyrrolo[4,3,2-de]pyrimido[4,5-C]pyridazine] (17).

### Example 8

### Synthesis of 5′-O-L-valyl-[6-Amino-4-methyl-8-(β-D-ribofuranosyl)pyrrolo[4,3,2-de]pyrimido[4,5-c]pyridazine] or 5′-O-L-valyl-TCN (scheme 9)

2′,3′-O-dilev TCN (7) (250 mg, 0.48 mmole), N-Boc L-valine (315 mg, 1.45 mmole), and DMAP (177 mg, 1.4 mmole) was dissolved in anhydrous DMF, DCC (1.45 mmole) in anhydrous DMF was added dropwise at room temperature. The solution turned cloudy within half hour. The reaction mixture was stirred for 20 hours after which the solvent was evaporated at 42° C. under high vacuum (20 tar) until dryness. The residue was dissolved in 50 ml ethyl acetate and consecutively washed with 40 ml saturated ammonium chloride, 40 ml water and 40 ml brine. After dried with Na₂SO₄ and removal of solvent, the residue was purified with flash silica gel column with DCM and MeOH (9:1) as eluent. The obtained syrup (18) was treated with 1.5 ml 2N hydrazine monohydrate in pyridine and acetic acid buffer for 10 minutes. After purifying with flash column (dichloromethane and methanol 9:1), the obtained white solid (19) was treated with 4M hydrochloric acid in dioxane for half hour. After washing with cold diethyl ether, the precipitated solid was further purified with preparative HPLC to obtain titled compound.

Mass Spectral data are: 420.1 (M+1). ¹H-NMR: δ 1.0 (m, 6H, two CH₃ of valyl), 2.35 (m, 1H, β CH of valyl), 3.49 (s, 3H, CH₃ of tricyclic ring), 3.82 (m, 2H, 2′&3′ CH), 4.1 & 4.39 (two multiple peaks, 2H, 5′ CH₂), 4.83 (m, 1H, 4′ CH), 5.45 (m, 1H, α CH of valyl) 6.02 (d, 1H, 1′CH), 7.39 & 8.05 (two singlet, 2H, two CH in tricyclic ring); (m is multiple peaks, and s is singlet peak).

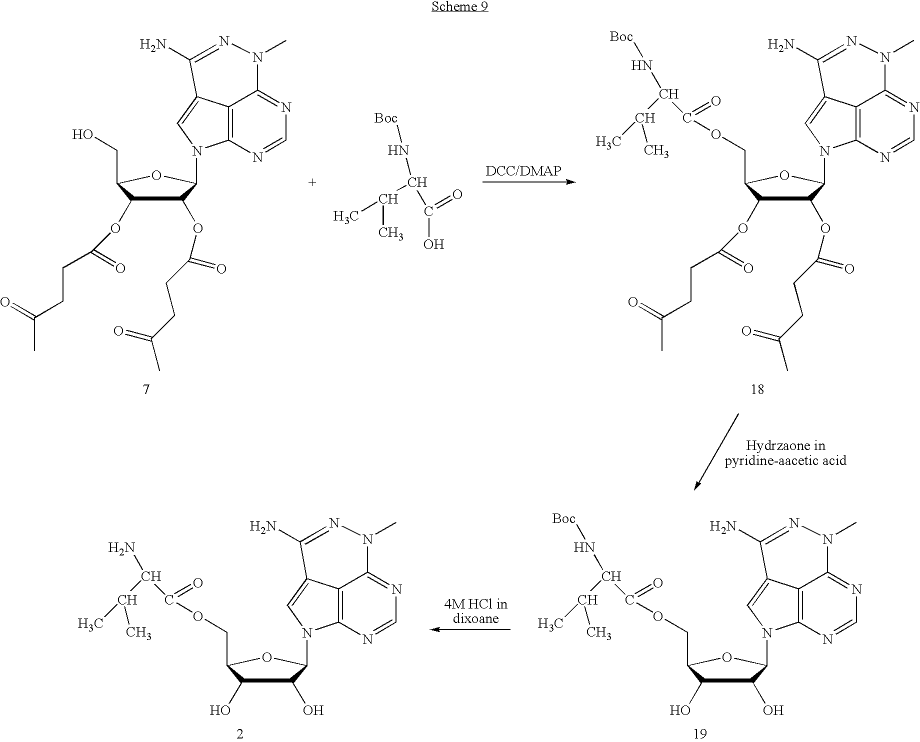

### Example 9

### Synthesis of [6-Amino-4-methyl-8-(β-D-ribofuranosyl)pyrrolo[4,3,2-de]pyrimido[4,5-c]pyridazine]-5′-[phenyl methoxy-L-valyl]-phosphate or 5′ O-L-valyl Phosphoramidate TCN (Scheme 10)

A solution of 0.67 ml (485 mg, 4.8 mmole) triethyl amine in 30 ml anhydrous dichloromethane was added dropwise within 1 hours to a mixture of L valine methyl ester (402 mg, 2.4 mmole) and phenyl dichlorophosphate (506 mg, 0.35 ml, 2.4 mmole) in 30 ml anhydrous dichloromethane at −78° C. After addition, the cooling bath was removed and the reaction temperature raise to room temperature within 2 hours. The reaction continued overnight. The solvent was evaporated under the protection of argon. 3 times of 10 ml of anhydrous diethyl ether was used to wash the solid residue. The ether solution was collected through filtration under protection of argon. After removal of ether, anhydrous tetrahydrofuran was used to dissolve the residue. This solution containing (3) was then added to THF solution of 2′,3′-O-dilev TCN (7) (261 mg, 0.5 mmole) with presence of N-methyl imidazole (0.4 ml, 414 mg, 0.5 mmole). After stirring at room temperature for 5 hours, the solvent was removed by rotavapor. The residue was purified by flash column with dichloromethane and methanol (96:4) as eluent to obtain 2′,3′-O-dilev-[6-Amino-4-methyl-8-(3-D-ribofuranosyl)pyrrolo[4,3,2-de]pyrimido[4,5-c]pyridazine]-5′-[phenyl methoxy-L-valyl]-phosphate (20).

2′,3′-O-dilev-[6-Amino-4-methyl-8-(β-D-ribofuranosyl)pyrrolo[4,3,2-de]pyrimido[4,5-c]pyridazine]-5-[phenyl methoxy-L-valyl]-phosphate (20) (250 mg, 0.3 mmole) was dissolved in 1 ml pyridine, 1 ml of 2N hydrazine monohydrate in pyridine-acetic acid buffer was added. After stirred at room temperature for 10 minute, the solution was directly subjected to a flash column with dichloromethane and methanol (9:1) as solvent to obtain titled compound 5′ O-L-valyl Phosphoramidate TCN (4).

Mass Spectral is 589.99 (M+1), ¹H-NMR (DMSO-d₆) δ: 0.85-0.92 (m, 6H, two CH₃ of valyl), 1.92-1.97 (m, 1H, β CH of valyl), 3.22-3.3 (m, 6H, CH₃ of tricyclic ring & CH₃ of methyl ester of valyl), 4.05-4.16 (m, 5H, 2′ and 3′ CH, 5′ CH₂, α CH of valyl), 4.25 (m, 1H, 4′ CH), 5.25 (broad band, 2H, 2′, 3′ OH), 5.95-5.98 (m, 2H, 1′ CH & α NH of valyl), 6.25 (broad band, 2H, NH₂ of tricyclic ring), 7.14-7.19 (m, 3H, three CH of phenyl), 7.22 (s, 1H, CH of tricyclic ring), 7.3-7.38 (m, 2H, two CH of phenyl), 8.13 (s, 1H, CH of tricyclic ring) (m is multiple peaks, and s is singlet peak).

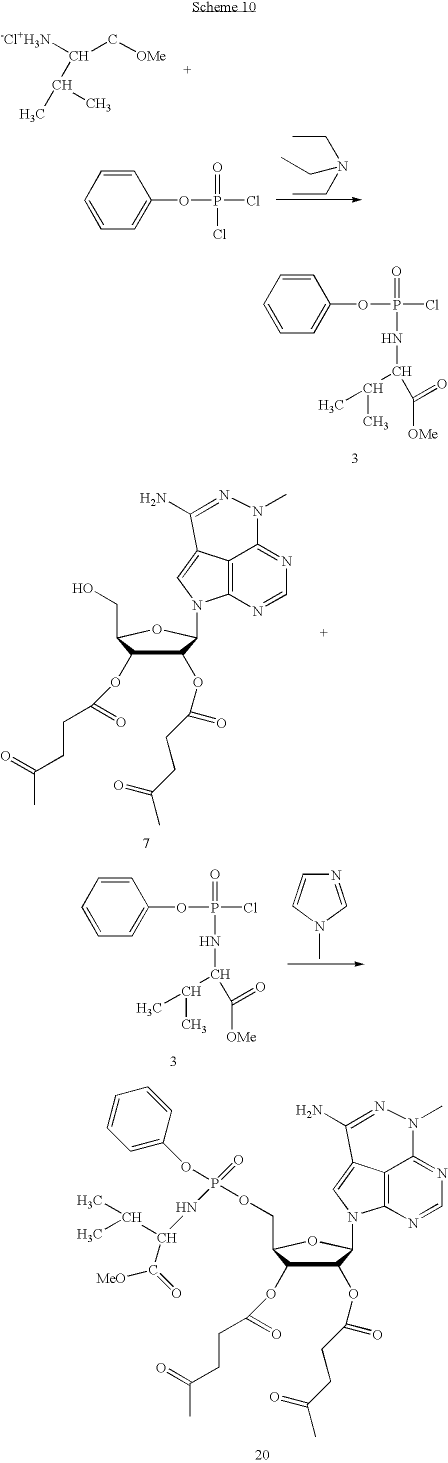

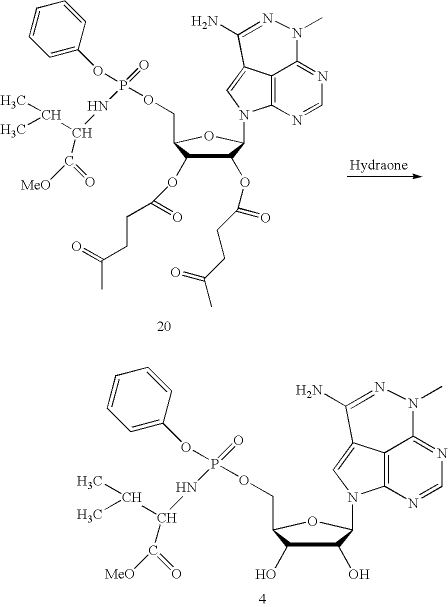

TABLE 3
Examples of TCN-based Amino Acid Prodrugs that are synthesized
by the methods described in the above EXAMPLES.

| | Amino Acid | Molecular | Estimated |
|---|---|---|---|
| Prodrug Designation | Promoiety | Weight | Log P¹ |
| 5′-O-D-isoleucyl TCN | D-ILE | 433.46 | 0.04 |
| 5′-O-L-isoleucyl TCN | L-ILE | 433.46 | 0.04 |
| 5′-O-D-valyl TCN | D-VAL | 419.44 | −0.37 |
| 5′-O-L-valyl TCN | L-VAL | 419.44 | −0.37 |
| 5′-O-glycyl TCN | GLY | 377.36 | −1.75 |
| 5′-O-D-phenylalanyl TCN | D-PHE | 467.48 | 0.42 |
| 5′-O-L-phenylalanyl TCN | L-PHE | 467.48 | 0.42 |
| 5′-O-D-leucyl TCN | D-LEU | 433.46 | −0.02 |
| 5′-O-L-leucyl TCN | L-LEU | 433.46 | −0.02 |
| 5′-O-D-alpha-aspartyl TCN | D-alpha ASP | 435.39 | −2.04 |
| 5′-O-L-alpha-aspartyl TCN | L-alpha ASP | 435.39 | −2.04 |
| 5′-O-D-beta-aspartyl TCN | D-beta ASP | 435.39 | −2.04 |
| 5′-O-L-beta-aspartyl TCN | L-beta ASP | 435.39 | −2.04 |

¹Estimated from Pro Log P program used in ChemDraw Pro, version 9.

### Example 10

Cytotoxicity of TCN, TCNP and their prodrugs. Cytotoxicity is tested in HFF cells cultured with the compounds over an 8 day period. Cytotoxicity was tested in HFF cells cultured with the compounds over an 8 day period. The cells were maintained in minimal essential medium (MEM) with Earle salts MEM(E)I supplemented with 10% fetal bovine serum in an atmosphere of 5% CO₂ at 37° C. At the start of the experiment, cells were plated in growth medium contain drug over a concentration range of 0 to 100 uM. The cells were maintained in these conditions for 8 days, then washed and scored for cytopathic effects. Cytopathology was scored by on a zero to 4 plus basis by visual inspection using 20 to 60 fold magnification. For the HFF cells, the TCNP is more toxic than TCN, and the two prodrugs show no cytotoxic effects (Table 1).

TABLE 4
Cytotoxicity of TCN, TCNP, and their
respective prodrugs in HFF cells.

| Compound | IC₅₀ (μM) |
|---|---|
| TCN | 100 |
| TCNP | 32 |
| 5′ O-valyl triciribine | >100 |
| 5′ O-valyl phosphoramidate triciribine | >100 |

### Example 11

### Determination of Binding Affinity of Amino Acid Prodrugs for the Intestinal Peptide Transporter

The TCN-based amino acid prodrugs are tested for their interaction with the dipeptide transporter, HPEPT1, using tissue culture cells that are engineered to overexpress HPEPT1. In this example, the cells that overexpress HPEPT1, termed DC5, are a human meduloblastoma cell line that is stably transfected with a eukaryotic expression vector encoding HPEPT1. In this assay, the ability of the prodrug to competitively inhibit the uptake of a known substrate of HPEPT1 is measured. The known substrate is the dipeptide Glycine-Sarcosine (Gly-Sar) that has a radioactive label. DC5 cells are plated at a density of 12,000 cells/well in 96-well tissue culture plates and allowed to grow for 2 days. The cells are washed once with 200 microliters of uptake buffer and aspirated. The plates are cooled to 4° C. and 25 ul of uptake buffer containing 125 nanomoles Gly-Sar (at a specific activity of 1 microcurie/micromole) is added. The uptake buffer also contains the prodrugs to be tested at concentrations ranging from 10 micromolar to 20 millimolar. The assay is initiated by placing the plate in a shaking water bath at 37° C. and is terminated after 10 min by rapid washing with multiple changes of 4° C. phosphate buffered saline (PBS). The radioactive Gly-Sar peptide that is transported by the hpept1 is extracted from the cell layer with 200 ul of a one to one mixture of methanol and water and is counted in 4 ml of CytoScint ES™ scintillation cocktail (ICN). The data are plotted as % Gly-Sar uptake of control (no competitive substrate) versus the competitive substrate concentration. The IC50, defined as that concentration which inhibits 50% of the uptake of the Gly-Sar uptake, indicates the degree of affinity that the test prodrug has for the hpept1. Typically, values that are below 10 mM indicate that the drug interacts with transporter. The data show that addition of HPEPT1 targeting promoieties to TCN and TCNP can improve the affinity of the drug for the HPEPT1 intestinal transporter.

### Example 12

### Determination of Prodrug Uptake mediated by Intestinal Transporter

Another aspect of the prodrug strategy for TCN and/or TCNP is to improve the absorption of the drug through the intestinal tract. In particular, the amino acid ester TCN-based prodrugs are designed to target transport of the prodrug to be mediated by nutrient transporters, e.g., the dipeptide transporter PepT1, that reside in the intestinal membrane. In these investigations, the test system for carrier-mediated uptake is HeLa cells that are transiently transfected with the human dipeptide transporter, hPept1. For the transient transfection, HeLa cells are seeded into multiwell plates at a density of 1×10⁵ cells/cm² and incubated for 24 hours. The cells are transfected with an expression plasmid containing the hPept1 gene under the control of the CMV promoter and are used for uptake studies within 48 hours of the transfection. A 1 mM prodrug solution in phosphate transport buffer, pH=6.0 is incubated for 45 minutes with HeLa/hPept1 cells two days post-transfection. The cells are lysed with a 1:1 mixture of MeOH and water, then centrifuged (20 min at 12,000×g), filtered and analyzed by HPLC. HeLa cells are used as a negative control. Protein amount of each sample is determined with the Bio-Rad Protein Assay using bovine serum albumin as a standard. Data are reported in nmole transported /mg of protein/45 minutes. The fold enhancement of the carrier mediated uptake is calculated from the HeLa cell control.

The ratio of the test versus control values provides a measure of uptake efficiency for the prodrug by the hpept1 transporter. The effect of stereochemistry on uptake efficiency is determined by comparing the uptake efficiency of D- and L-amino acid-containing prodrugs.

### Example 13

Testing of the Amino Acid Ester-containing Prodrugs for Activation with a prototype Activation Enzyme-BPHL

To look at activation of the amino acid prodrug (e.g., the removal of the amino acid ester), the prodrugs are tested for hydrolysis using the prototype activation enzyme-purified biphenyl hydrolase-like enzyme (BPHL) (Kim I, et al. (2003). J Biol Chem. 278, (28): 25348-56). A solution containing 1 mM of each compound is incubated with the enzyme at 25° C. The reaction is stopped by the addition of 5% trifluoroacetic acid and the amount of parent compound is determined by HPLC analysis. Valacyclovir (VACV) hydrolysis by BPHL is used as a control. The prodrugs of the invention are compared to one another in order to determine the effect of the structure of the promoiety (e.g. valyl v. lysyl), as well as the position of the linkage to TCN and TCNP moiety (e.g. the positions corresponding to the 2′ v. 3′ v. 5′ hydroxyls of TCN and/or TCNP), as substrates for the hydrolytic activity of the BPHL enzyme.

### Example 14

### Testing for Activation of the Prodrugs of the Invention with Intestinal Cell Lysates and Plasma

Confluent Caco-2 cells are washed with phosphate buffer saline (PBS, pH 7.4) and are harvested with 0.05% Trypsin-EDTA at 37° C. for 5-10 min. Trypsin is neutralized by adding DMEM. The cells are washed off the plate and spun down by centrifugation. The pelleted cells are washed twice with pH 7.4 phosphate buffer (10 mM), and resuspended in pH 7.4 phosphate buffer (10 mM) to obtain a final concentration of approximately 4.70×10⁶ cells/mL. The cells are lysed with one volume 0.5% Triton-X 100 solution. The cell lysate is homogenized by vigorous pipetting and total protein is quantified with the BioRad DC Protein Assay using bovine serum albumin as a standard. The hydrolysis reactions are carried out in 96-well plates (Corning, Corning, N.Y.). Caco-2 cell suspension (230 μl) is placed in triplicate wells and the reactions are started with the addition of substrate and incubated at 37° C. At various time points, 40 μL aliquots are removed and added to two volumes of 10% ice-cold TFA. The mixtures are centrifuged for 10 mM at 1800 ref and 4° C. and the supernatant filtered through a 0.45 μm filter. The recovered filtrate is analyzed by HPLC. The estimated half lives (t_{1/2}), obtained from linear regression of pseudo-first-order plots of prodrug concentration vs time are compared. The effects noted include: a) the effect of structure of promoiety on stability, and where applicable, b) the effect of stereochemistry of the promoiety on the stability of the prodrugs.

### Example 15

Solution and Biological Homogenate Stability of 5′-O-amino Acid Prodrugs of TCN

The chemical stabilities of the TCN prodrugs are determined in pH 7.4 phosphate buffer (10 mM) at 37° C. in order to obtain the contribution of non-enzymatic hydrolysis. The hydrolysis reactions are carried out in 96-well plates. PBS buffer (230 microliters) is placed in triplicate wells and the reactions are started with the addition of TCN prodrugs and incubated at 37° C. At various time points, 40 microliter aliquots are removed and added to 40 microliter of 10% ice-cold TFA. The mixtures are centrifuged for 10 min at 2000×g at 4° C. then filtered through a 0.45 micron filter. The filtrate is analyzed by HPLC and mean t_{1/2} are determined.

To test stability in human plasma, 230 μL undiluted plasma is added to each well in triplicate and 40 μL of substrate is added to start the reactions which are conducted at 37° C. for up to 4 hours. At various predetermined time points, 40 μl aliquots are removed and added to two volumes of 10% ice-cold TFA. The mixtures are centrifuged for 10 min at 1800 rcf at 4° C. and the supernatant is filtered through a 0.45 μm filter. The recovered filtrate is analyzed by HPLC. Half-live (t_{1/2}) estimates are obtained from linear regression of pseudo-first-order plots of prodrug concentration vs time. The half life values of the TCN and TCNP prodrugs in plasma are compared to that in phosphate buffer, pH 7.4; are determined and compared and in Caco-2 cell homogenates. These enhanced rates of degradation suggest specific enzymatic action. The effects noted include: a) the effect of structure of promoiety on stability, and b) the stereochemistry of the promoiety affected the stability of the prodrugs.

Stability of the TCN-based prodrugs in Caco-2 homogenates is illustrated in this example. Homogenates are prepared by adding an equal volume of 0.5% Triton-X 100 to 5×10⁶ Caco-2 cells/mL followed by vigorous vortexing. The hydrolysis experiments are carried out in triplicate as described above in chemical stability section using cell homogenate instead of buffer. The rate of hydrolysis of all prodrugs tested is determined as t_{1/2} (min.) and compared in the Caco-2 homogenates in buffer alone; higher rates in the homogenates vs. buffer indicating enzymatic conversion of the prodrug to TCN or TCNP.

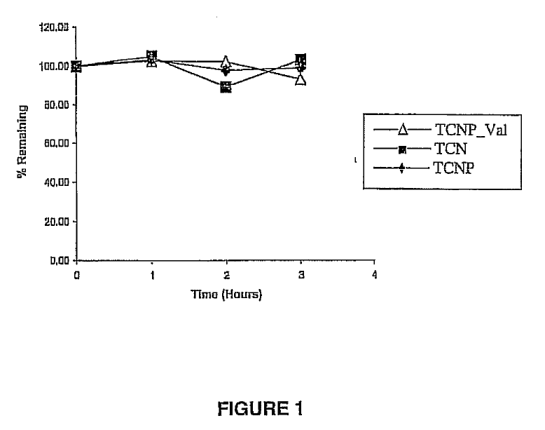

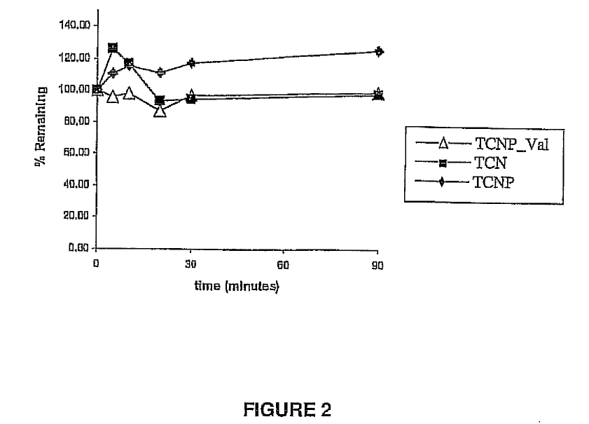

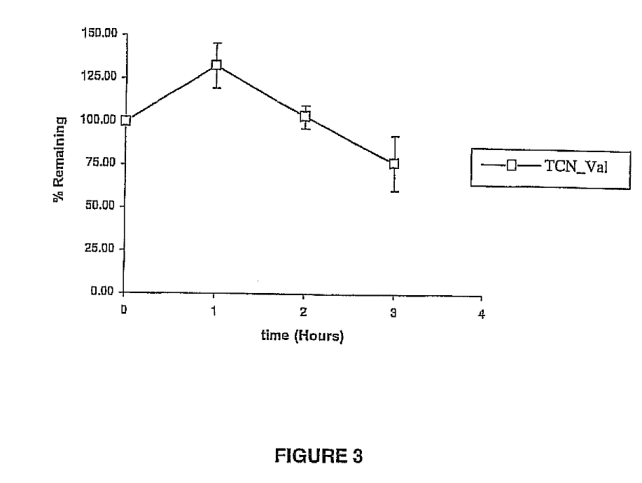

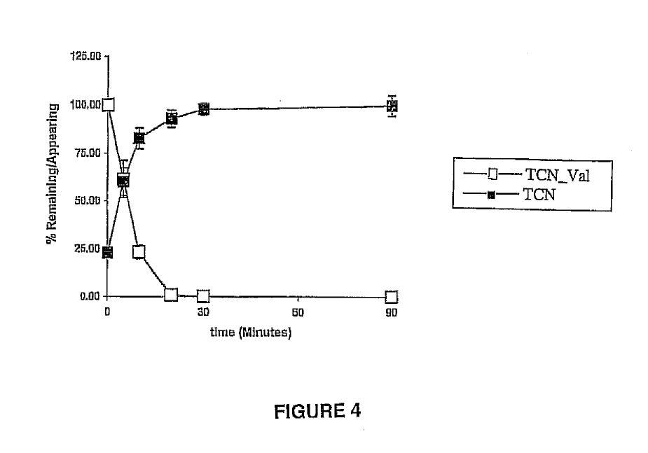

Stability of the prodrugs O-valyl TCN and O-valyl TCNP, and their parent compounds was assessed in 100 mM phosphate buffer, pH 6.5 and in rat liver homogenates at 37° C. For the buffer stability, timed aliquots are taken at 0, 1, 2, and 3 hours, mixed with cold 10% TCA and analyzed for prodrug and parent compound by LC/MS/MS. For the liver homogenates, samples are taken at 0, 5, 10, 20, 30 and 90 minutes, mixed with cold 10% TCA, then centrifuged for 10 minutes to remove precipitated liver homogenate protein. Samples are analyzed be LC/MS/MS. It can be seen in FIGS. 1 and 2 that the TCN, TCNP and 5′ O-valyl phosphoramidate triciribine are stable in buffer and liver homogenates. In contrast, the 5′ O-valyl triciribine prodrug (TCN_Val) shows slight instability in buffer, and marked instability in liver homogenates, with a t_{1/2} of approximately 12 minutes in the liver homogenates (FIGS. 3 and 4).

### Example 16

### Caco-2 Monolayer Transport and Stability Studies Using TCN-Based Prodrugs

The transepithelial transport of the prodrugs of the invention including 5′ O-valyl TCN and 5′ O-valyl phosphoramidate TCN is tested in Caco-2 monolayers grown in transwell filters following standard procedures as described in detail in Landowski, C. P. et al., Pharm Res, 2005. 22(9): p. 1510-8. In these experiments, transport studies are performed 21 days post-seeding. The assay is initiated by adding drug transport solution (0.2 mM drug in MES buffer, pH 6.0 containing 5 mM D-glucose, 5 mM IVIES, 1 mM CaCl2, 1 mM MgCl₂, 150 mM NaCl, 3 mM KCl, 1 mM NaH2PO4) to the apical chamber of the Caco-2 insert. Two hundred microliter aliquots are withdrawn from the basolateral chamber at predetermined intervals and replaced with fresh HEPES pH 7.4 buffer. The epithelial integrity of representative cell monolayers is assessed by monitoring transepithelial resistance. Permeabilities of the parent and prodrugs are determined and compared. The stability of the drugs in both chambers of the insert is also monitored.

### Example 17

As illustrative of the enhanced oral absorption after oral dosing with the TCN prodrugs, TCN, TCNP, 5′ O-valyl TCN and 5′ O-valyl phosphoramidate TCN are individually administered to the duodenum of a rat and systemic plasma concentrations are determined for 4 hours after administration. For these studies, male albino Sprague-Dawley rats, 9-10 weeks old and weighing 250-350 g are fasted for 18 hours with free access to water. The rats are anesthetized with 2-5% isofluorane. To withdraw timed plasma samples, a catheter is placed in the jugular vein. The abdomen is opened by a 4-5 cm midline incision and the duodenal segment is located. 0.5 ml of a 6 mg/ml drug solution (or suspension) is injected directly into the duodenal segment, the intestine is placed back into the abdominal cavity and the incision is covered with gauze. Plasma samples (˜0.5 ml) are withdrawn over a 4 hour period and the systemic plasma concentrations of the injected prodrug and/or parent compounds are determined simultaneously using an LC/MS/MS method described below.

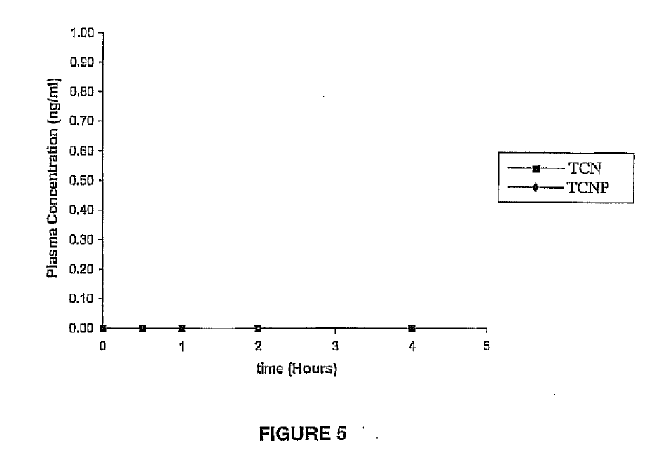

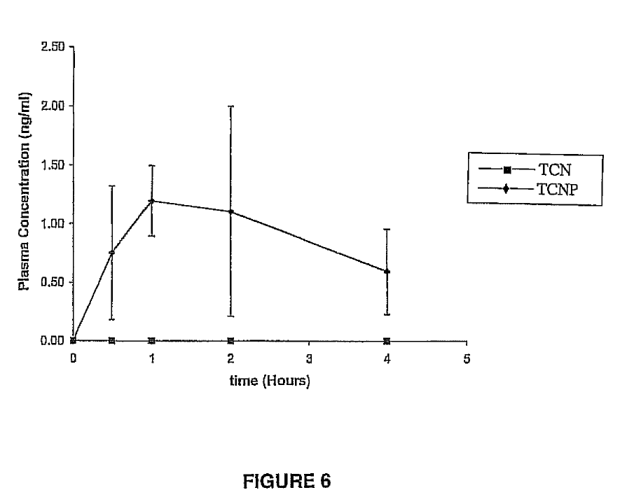

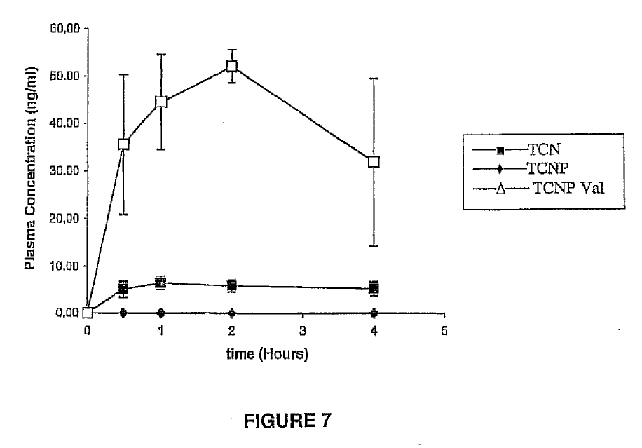

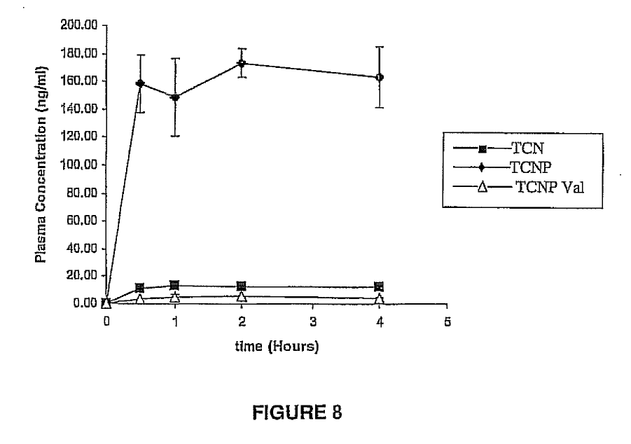

As shown in FIGS. 5 to 8, it can be seen that for TCN and TCNP there is very little intestinal absorption of the compounds, with peak plasma levels only reaching about 1.25 ng/ml. In contrast, the prodrug compounds show much greater levels in plasma. Dosing with the two prodrugs results in more complex Cp×time curves, since both of the prodrugs are hydrolyzed, at least in part, to their parent compound.

The 5′ valyl phosphoramidate TCN prodrug shows the greatest absorption of the two prodrugs and the major detectable compound is the parent compound, TCNP, which has a Cmax of 173.6 ng/ml. Total exposure to TCN containing compounds, as measured by the AUC0-4 hr, also shows that the phosphoramidate linked prodrug has the greatest potential for oral absorption. These results are summarized in Table 5.

TABLE 5
PK parameters of Cmax and AUC after duodenal administration
of TCN, TCNP and their respective prodrugs.

| | Cmax | AUC_{0-4 hr} |
|---|---|---|
| Compound | (ng/mL) | (ng/ml)/hr |
| TCN | Below | ND |
| | detection limit | |
| TCNP | 2.2 | 4.4 ± 0.9 |
| 5′ O-valyl triciribine | | |
| prodrug | 52.2 | 161 ± 33.7 |
| TCN | 6.4 | 21.5 ± 4.8 |
| 5′ valyl phosphoramidate TCN | | |
| prodrug | 5.3 | 17.4 ± 2.5 |
| TCNP | 173.6 | 615.1 ± 11.4 |
| TCN | 13.4 | 47.7 ± 2.2 |

### Example 18

For analysis by LC/MS/MS, samples are subjected to solid phase extraction using Waters Cation SPE cartridges (MCX). The cartridges are conditioned with 1 mL methanol and 1 mL water. 0.25 mL samples are treated with an equal volume of 2% phosphoric acid and loaded onto the SPE column. The column is washed with 1 mL of 1% TFA methanol solution and 1 mL of methanol then eluted with 1 mL of 2% NH4OH/methanol. The eluate is evaporated to dryness under a nitrogen stream in a Turbovap solution evaporator and reconstituted in 0.25 mL of HPLC mobile phase. This material is transferred to microinserts and analyzed.

Ten microliter aliquots of the reconstituted sample are separated on a C18, 2.2 mm×10 cm column (Higgins Analytical), at a flow rate of 0.2 ml/min over a run time of 3 minutes. The mobile phase for the separation consists of an isocratic gradient using the following mobile phases A) 70% 0.1% formic acid in water to B) 30% acetonitrile. The MS/MS detector is run under MRM positive acquisition mode, with a cone voltage of 30 Volts, a collision energy of 5 Volts with a collision gas pressure of 1×10-3 mbar.

### Example 19

### Solubility Testing of TCN-Based Prodrugs

Solubility of parent and prodrugs in water is tested by known methods; for example by dropwise addition of water to 5 mg of parent and prodrug with continuous stirring and determining the concentration at which the parent and prodrug are dissolved. In this manner, it is determined whether the addition of the promoiety to the parent compound enhances the aqueous solubility.

### Example 20

### Expeditious Synthesis of TCN

An improved synthesis of triciribine is provided in Scheme 11 provided below that contrasts with the prior art ten-step synthesis of TCN. Anthony R. et al. Nucleosides, Nucleotides & Nucleic Acids 2004, Vol. 23, Nos. 1 & 2, pp. 31-39. The steps of the improved synthesis are carried out under an inert atmosphere of nitrogen and in suitable inert solvent under reaction conditions that are comparable to those used in Porcari et al. The TCN so produced is characterized and confirmed by NMR.

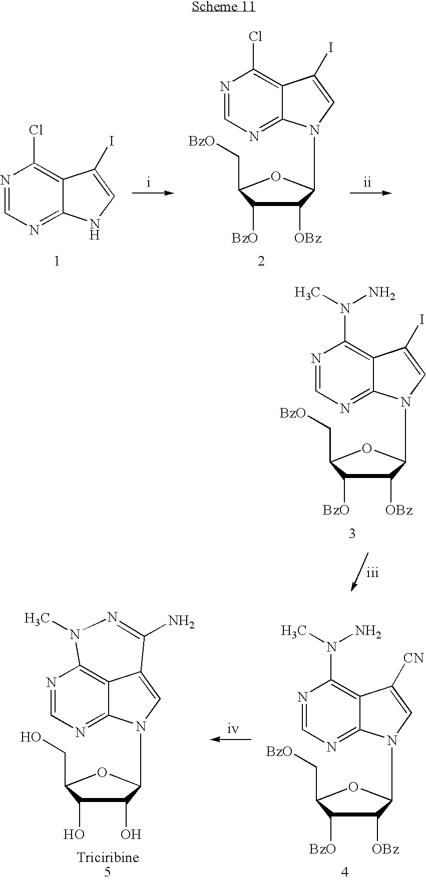

i): N,O-Bis(trimethylsilyl)acetamide, 1-O-acetyl-2,3,5-tri-O-benzoyl-beta-D-ribofuranose, trimethylsilyl trifluoromethanesulfonate.
ii): methylhydrazine,
iii): zinc cyanide, tetrakis(triphenylphosphine)palladium (0),

### vi): NaOMe/MeOH

### Example 21

### Facile Protection of 2′ and 3′ Hydroxyl Groups of TCN

A 2′ and 3′ hydroxyl protected analog of TCN is produced according to Scheme 12 in which abbreviated compounds have the conventional meanings in the art and as used throughout the specification. The protecting groups on the 2′ and 3′ hydroxy positions of TCN are facile and removable upon formation of an otherwise desired inventive TCN prodrug.

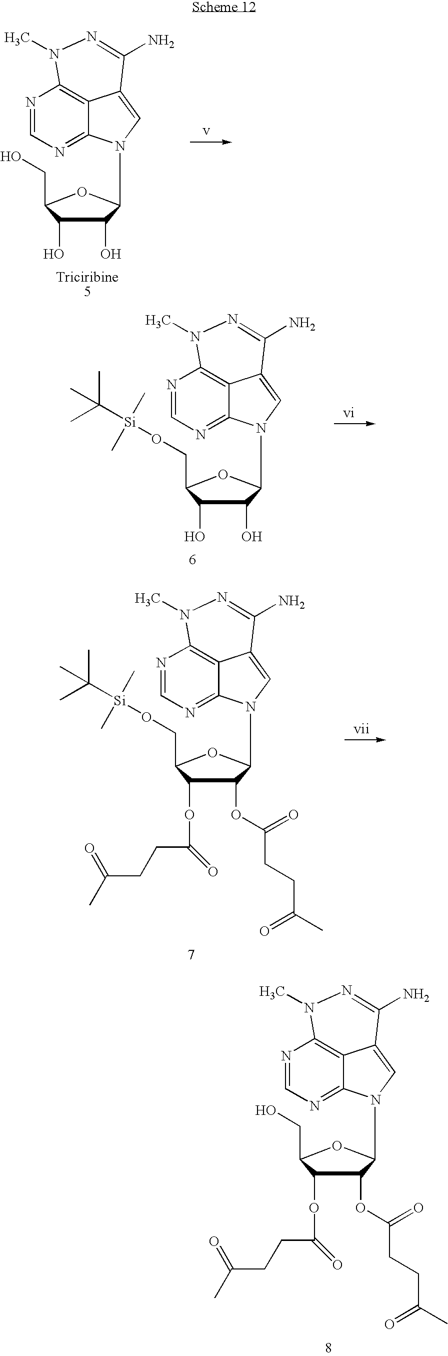

v): Tert-butyldimethylsilyl chloride and imidazole in DMF,
vi): Levulinic acid, DCC, DMAP in ethyl acetate,
vii): TBAF-acetic acid (1:2 mole ratio) in tetrahydrofuran

### Example 22

### Synthesis of D and L Valyl Phosphoramidate Prodrug of Triciribine

Reaction Scheme 13 affords 5′ O-valyl-phosphoramidate TCN incorporating either L or D valine isomer, or a racemate thereof, depending on the valine isomer precursor. Reaction Scheme 13 is noted to track Scheme 3 of Example 3 with the addition of the deprotection of 2′ and 3′ TCN hydroxyl sites to afford the title prodrug.

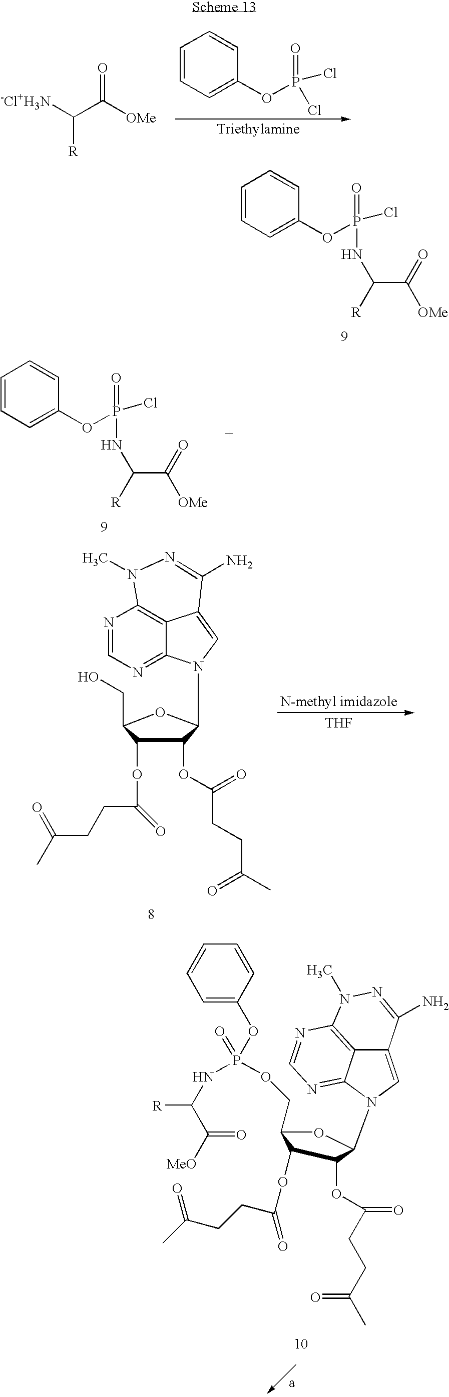

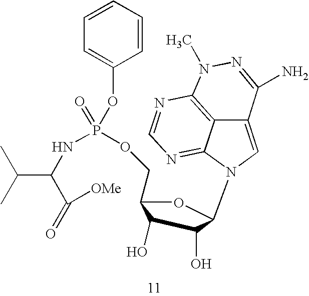

viii: 1 ml 2M hydrazine hydrate in pyridine-acetic acid buffer

Any patents or publications mentioned in this specification are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The apparatus and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A composition, comprising:
a prodrug having the structural formula: 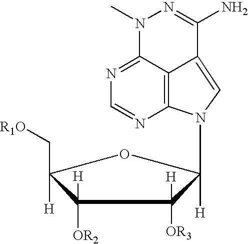
where R₁, R₂ and R₃ are each independently H, or selected from the group consisting of: an amino acid, a dipeptide, a tripeptide, and z 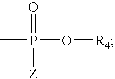
where Z and at least one of R₁, R₂ and R₃ is an amino acid, a dipeptide or a tripeptide
where R₄ is aliphatic, aryl, or heteroaryl;
or a salt or hydrate thereof.

2. The composition of claim 1, where the amino acid is an L-amino acid.

3. The composition of claim 1, where the amino acid is a D-amino acid.

4. The composition of claim 1, where R₄ is benzyl.

5. The composition of claim 1, where at least one of R₁, R₂ and R₃ is selected from the group consisting of:
-D-isoleucyl; -L-isoleucyl; -D-valy; -L-valyl ; -glycyl; -D-phenylalanyl; -L-phenylalanyl; -D-leucyl; -L-leucyl; -L-aspartyl; -D-alpha-aspartyl; -L-alpha-aspartyl; -D-beta-aspartyl; -L-beta-aspartyl; and -L-prolyl; -D-isoleucyl phosphoramidate; -L-isoleucyl phosphoramidate; -D-valyl phosphoramidate; -L-valyl phosphoramidate; -glycyl phosphoramidate; -D-phenylalanyl phosphoramidate; -L-phenylalanyl phosphoramidate; -D-leucyl phosphoramidate TCN; 5′-O-L-leucyl phosphoramidate TCN; 5′-O-L-aspartyl phosphoramidate; -D-alpha-aspartyl phosphoramidate; -L-alpha-aspartyl phosphoramidate; D-beta-aspartyl phosphoramidate; -L-beta-aspartyl phosphoramidate; and -L-prolyl phosphoramidate.

6. The composition of claim 1, where R₁ is selected from the group consisting of: an amino acid, a dipeptide a tripeptide, and 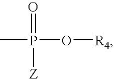
where R₂, and R₃ are each independently H, an amino acid, a dipeptide, a tripeptide or

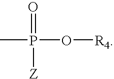

7. The composition of claim 1, where at least one of R₁, R₂, R₃, and 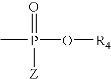
is a substrate for a transporter.

8. The composition of claim 7, where the transporter is an intestinal transporter.

9. The composition of claim 7, where the transporter is selected from HPEPT1, and HPT1.

11. The composition of claim 1, characterized by at least three-fold greater bioavailability compared to TCN or TCNP.

12. The composition of claim 1 further comprising at least one neoplastic agent selected from the group consisting of: floxuridine, gemcitabine, cladribine, decarbazine, melphalan, mercaptopurine, thioguanine, cis-platin, and cytarabine.

13. A composition, comprising:
a prodrug having the structural formula: 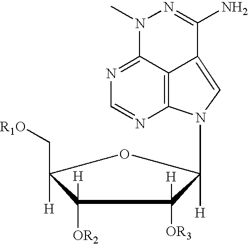
where R₁ is an amino acid or 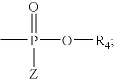
R₂ and R₃ are both H; and Z is an amino acid.

14. The composition of claim 13, wherein the amino acid is a non-polar amino acid.

15. The composition of claim 13, wherein the amino acid is a substrate for an intestinal transporter.

16. A composition of claim 13, wherein the amino acid is an L-amino acid.

17. The composition of claim 13, wherein the amino acid has the formula HOOC—(CH₂)ₙ—CH(NH₂)—COOH where n is an integer in the range of 1-6, inclusive.

18. The composition of claim 13, wherein the prodrug is selected from the group consisting of: 5′-O-D-isoleucyl TCN; 5′-O-L-isoleucyl TCN; 5′-O-D-valyl TCN; 5′-O-L-valyl TCN; 5′-O-glycyl TCN; 5′-O-D-phenylalanyl TCN; 5′-O-L-phenylalanyl TCN; 5′-O-D-leucyl TCN; 5′-O-L-leucyl TCN; 5′-O-L-aspartyl TCN; 5′-O-D-alpha-aspartyl TCN; 5′-O-L-alpha-aspartyl TCN; 5′-O-D-beta-aspartyl TCN; 5′-O-L-beta-aspartyl TCN; and 5′-O-L-prolyl TCN 5′-O-D-isoleucyl phosphoramidate TCN; 5′-O-L-isoleucyl phosphoramidate TCN; 5′-O-D-valyl phosphoramidate TCN; 5′-O-L-valyl phosphoramidate TCN; 5′-O-glycyl phosphoramidate TCN; 5′-O-D-phenylalanyl phosphoramidate TCN; 5′-O-L-phenylalanyl phosphoramidate TCN; 5′-O-D-leucyl phosphoramidate TCN; 5′-O-L-leucyl phosphoramidate TCN; 5′-O-L-aspartyl phosphoramidate TCN; 5′-O-D-alpha-aspartyl phosphoramidate TCN; 5′-O-L-alpha-aspartyl phosphoramidate TCN; 5′-O-D-beta-aspartyl phosphoramidate TCN; 5′-O-L-beta-aspartyl phosphoramidate TCN; and 5′-O-L-prolyl phosphoramidate TCN.

19. A method of treatment of a subject, comprising:
administering to a subject in need thereof a therapeutically effective amount of a composition comprising a prodrug having the structural formula: 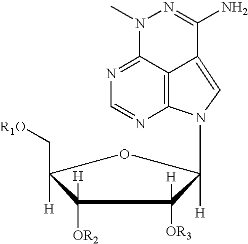
where R₁, R₂ and R₃ are each independently H, or selected from the group consisting of: an amino acid, a dipeptide, a tripeptide, and 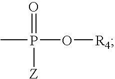
where Z and at least one of R₁, R₂ and R₃ is an amino acid, a dipeptide or a tripeptide ;
where R₄ is aliphatic, aryl, or heteroaryl;
and a pharmaceutically acceptable carrier.

20. The method of claim 19, wherein the subject is human.

21. The method of claim 19, wherein the administration is oral administration.

22. The method of claim 19, wherein the subject has or is at risk of having cancer.

23. The method of claim 21, where the oral administration is subsequent to parenteral administration of at least one of TCN and TCNP.

24. The method of claim 21, wherein the subject has a disorder characterized by overexpression of AKT in a tissue of said subject, and wherein said oral administration detectably increases apoptosis in said tissue.

25. The method of claim 24, wherein the tissue is a tumor.

26. Use of a composition according to claim 1, in preparing an anticancer medicament.

27. An anticancer method substantially as described.

28. Use of a composition according to claim 1, in preparing an anticancer medicament.

29. An anticancer method substantially as described.

30. A composition substantially as described.

31. A method of synthesizing a prodrug substantially as described.
